SEQUENCE LISTING (23 sequences)

SEQ ID NO: 1 - protein, 280 aa, synthetic construct
  REGION 1..280: note = Synthetic sequence
MQAKYSSTRDMLDDDGDTTMSLHSQGSATTRHPEPRRTEHRAPSSTWRPVALTLLTLCLVLLIGLAALGLLFFQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 2 - protein, 207 aa, synthetic construct
  REGION 1..207: note = Synthetic sequence
QYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 3 - protein, 227 aa, synthetic construct
  REGION 1..227: note = Synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 227 aa, synthetic construct
  REGION 1..227: note = Synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 449 aa, synthetic construct
  REGION 1..449: note = Synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 6 - protein, 227 aa, synthetic construct
  REGION 1..227: note = Synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 226 aa, synthetic construct
  REGION 1..226: note = Synthetic sequence
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, synthetic construct
  REGION 1..227: note = Synthetic sequence
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 226 aa, synthetic construct
  REGION 1..226: note = Synthetic sequence
YGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 10 - protein, 226 aa, synthetic construct
  REGION 1..226: note = Synthetic sequence
YGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSPGK

SEQ ID NO: 11 - protein, 226 aa, synthetic construct
  REGION 1..226: note = Synthetic sequence
YGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSPGK

SEQ ID NO: 12 - protein, 448 aa, synthetic construct
  REGION 1..448: note = Synthetic sequence
YGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSPGKGGGGSGGGGSGGGGSQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 13 - protein, 15 aa, synthetic construct
  REGION 1..15: note = Synthetic sequence
GGGGSGGGGSGGGGS

SEQ ID NO: 14 - protein, 20 aa, synthetic construct
GGGGSGGGGSGGGGSGGGGS

SEQ ID NO: 15 - protein, 10 aa, synthetic construct
GGGGSGGGGS

SEQ ID NO: 16 - protein, 5 aa, synthetic construct
GGGGS

SEQ ID NO: 17 - protein, 4 aa, synthetic construct
GGGS

SEQ ID NO: 18 - protein, 15 aa, synthetic construct
GGGSSGGGSSGGGSS

SEQ ID NO: 19 - protein, 187 aa, Synthetic construct
GNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 20 - protein, 10 aa, synthetic construct
DKTHTCPPCP

SEQ ID NO: 21 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 22 - protein, 107 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 449 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

(19) United States
(12) Patent Application Publication
(10) Pub. No.: US 2026/0035434 A1
(43) Pub. Date: Feb. 5, 2026

(54) HETERODIMERIC FC-CLEC-1 FUSION MOLECULE AND USES THEREOF

(71) Applicant: OSE IMMUNOTHERAPEUTICS, NANTES (FR)

(72) Inventors: Nicolas POIRIER, GRANDCHAMPS DES FONTAINES (FR); Virginie THEPENIER, SAINTE-PAZANNE (FR); Geraldine TEPPAZ, SAINT HERBLAIN (FR); Julien TAURELLE, NANTES (FR)

(21) Appl. No.: 18/996,370

(22) PCT Filed: Aug. 1, 2023
(86) PCT No.: PCT/EP2023/071329

§ 371 Date: Jan. 17, 2025

(30) Foreign Application Priority Data
Aug. 1, 2022 (EP) 22306163.1

(51) Int. Cl. C07K 14/705; A61K 38/00; A61P 35/00; C07K 16/30; C12N 15/62

(52) U.S. Cl. CPC C07K 14/7056; A61K 38/00; A61P 35/00; C07K 16/30; C12N 15/62; C07K 2317/524; C07K 2317/526; C07K 2317/53; C07K 2317/56; C07K 2319/30

(57) ABSTRACT

The present invention concerns a heterodimeric Fc-CLEC1 fusion molecule comprising at least one binding moiety comprising at least a portion of the extracellular domain of CLEC-1, and an immunoglobulin fragment comprising a heterodimeric Fc region comprising two different Fc polypeptide chains. The invention provides heterodimeric Fc-CLEC1 fusion molecules that have improved capabilities regarding their toxicity, half-live, bioavailability, as compared to prior art fusion molecule binding to CLEC-1. The present invention also relates to the use of the fusion molecule in therapy.

### FIELD OF THE INVENTION

The present invention concerns a heterodimeric Fc-CLEC1 fusion molecule comprising at least one binding moiety comprising at least a portion of the extracellular domain of CLEC-1, and an immunoglobulin fragment comprising a heterodimeric Fc region comprising two different Fc polypeptide chains. The invention provides heterodimeric Fc-CLEC1 fusion molecules that have improved capabilities regarding their toxicity, half-live, bioavailability, as compared to prior art fusion molecule binding to CLEC-1. The present invention also relates to the use of the heterodimeric Fc-CLEC1 fusion molecules in therapy.

### BACKGROUND OF THE INVENTION

One of the issues in the treatment of cancer is to destroy tumor cells without damaging healthy tissue. Anti-tumoral immunotherapies gained an increasing interest in the past decade as they redirect the patient's immune system to destroy cancer cells. It has been clearly demonstrated that tumor cells exploit immune checkpoints to escape immune defenses. The critical roles of these inhibitory checkpoints in obstructing anti-tumor immunity have been illustrated by the remarkable success of antibody-driven blockade of T-cell immune checkpoints. However, a significant proportion of patients do not respond or develop resistance to these checkpoint inhibitor (CKI) therapies, generally after classical chemotherapy failure. Effective novel treatments are needed for these patients, and myeloid cells represent promising therapeutic target. Indeed, myeloid cells are the most abundant tumor infiltrating immune cells. C-type lectins (CLEC) expressed on immune cells are sensors of the environment and immune response modulators. CLEC-1 represents a potential therapeutic target to boost myeloid cells and anti-tumor response. To better decipher CLEC-1 function and develop compounds aimed at modulating its activity, there is a need for molecules mimicking the effect of CLEC-1, possibly with improved capabilities, to enhance or stimulate signaling pathways and/or cells expressing CLEC-1.

CLEC-1 (also designated CLEC-1A, or CLEC1, or CLEC1A) is a C-type lectin-like receptor, i.e., C-type lectin-like receptor-1, that is in particular expressed in mammal species, more particularly in human. More precisely, CLEC-1 belongs to the DECTIN-1 cluster of C type-lectin like receptors (CTLRs) that also includes CLEC-2, DECTIN-1 (CLEC7-A), CLEC-9A, MICL, MAH and LOX-1 (Colonna M, Samaridis J, Angman L. Molecular characterization of two novel C-type lectin-like receptors, one of which is selectively expressed in human dendritic cells. Eur J Immunol. 2000; 30 (2): 697-704). Functionally, C-type lectin receptors (CLRs) are a large family of transmembrane and soluble receptors that contain one or more carbohydrate-recognition domain able to recognize a wide variety of glycans on pathogens or on self-proteins. For these receptors, glycan recognition is dependent from Ca²⁺. Many related-CLRs are nonetheless able to recognize carbohydrates but independently of Ca²⁺; these receptors are referred to C-type lectin-like receptors (CTLRs), a family of receptors encompassing CLEC-1. These receptors are of particular interest for their role in coupling both innate and adaptive immunity. CTLRs are expressed mostly by cells of myeloid lineage such as monocytes, macrophages, dendritic cells (DCs), and neutrophils. CTLRs not only serve as antigen-uptake receptors for internalization and presentation to T cells but also trigger multiple signaling pathways leading to NF-κB, type I interferon (IFN), and/or inflammasome activation. By their capacity to present antigen and ensure the balance between cellular activation and suppression, CTLRs have emerged as challenging pharmacological targets to treat a wide variety of diseases including cancers, autoimmune diseases or allergy. CTLR modulation seems to represent a promising strategy for disease management although attempts at identifying ligands as well as efforts to elucidate their role in immunity remain incomplete to date.

CLEC-1 is known to be expressed on myeloid and endothelial cells. CLEC-1 has been described as a receptor that may be up-regulated by immunomodulatory mediators and that may moderate T cell activation (Thebault P. et al The C-Type Lectin-Like Receptor CLEC-1, Expressed by Myeloid Cells and Endothelial Cells, Is Up-Regulated by Immunoregulatory Mediators and Moderates T Cell Activation. J Immunol 2009; 183:3099-3108). The present inventors showed that CLEC-1 is expressed at the cell-surface by conventional DCs (cDCs) and by small subsets of monocytes and DCs in human blood and is enhanced by the immunosuppressive cytokine TGFβ (see international application No. WO2018073440). They demonstrated in both rodent and human that CLEC-1 acts as an inhibitory receptor in myeloid cells and prevent IL12p40 expression and downstream Th1 and Th17 in vivo responses (Lopez-Robles M D et al., Cell-surface C-type lectin-like receptor CLEC-1 dampens dendritic cell activation and downstream Th17 responses Blood Adv. 2017; March 22;1 (9)557-568).

These properties provide interest to consider CLEC-1 in the design of additional means for the treatment of health conditions that involve the immune response of the patient, in particular to develop new treatment against cancer. In particular, these results call for additional research in order to provide a compound able to bind to at least one ligand of CLEC-1 and/or to trap at least one ligand of CLEC-1 with enhanced properties, such as better half-life, or enhanced functions, such as complement-dependent cytotoxicity (CDC), antibody-dependent cellular cytotoxicity (ADCC) and antibody-dependent cell phagocytosis (ADCP).

### DESCRIPTION OF THE INVENTION

The present invention relies on the association within a fusion molecule of a binding moiety comprising or consisting of at least a portion of the extracellular domain of CLEC-1, either mutated or not as compared to wild type CLEC-1, and a heterodimeric Fc compound, comprising at least 2 different Fc polypeptide chains.

As will be detailed further in the application, the inventors provide evidence that the fusion molecule of the invention leads notably to molecules with at least one of the following properties:
a heterodimeric Fc-CLEC-1 fusion molecule with a reduced in vivo toxicity as compared to prior art anti-CLEC-1 compounds, notably prior art Fc-CLEC-1 fusion proteins;
a heterodimeric Fc-CLEC-1 fusion molecule that does not form aggregates (e.g. an accumulation of fusion molecules clumped together and forming a body or a mass; aggregates can be considered when more than two fusion proteins are accumulated and clumped together-in the context of this invention, a dimer of fusion proteins cannot be considered as an aggregate),
a heterodimeric Fc-CLEC-1 fusion molecule that has a prolonged half-life, notably as compared to prior art anti-CLEC-1 compounds, in particular prior art Fc-CLEC-1 fusion proteins;
a heterodimeric Fc-CLEC-1 fusion molecule that has an enhanced bioavailability as compared to prior art anti-CLEC-1 compounds, in particular prior art Fc-CLEC-1 fusion proteins;
a heterodimeric Fc-CLEC-1 fusion molecule that does not lead to an increase in the secretion of pro-inflammatory cytokines, in particular an increase in the secretion of IL-6.

As illustrated in the examples of the invention, the heterodimeric Fc-CLEC-1 fusion molecules of the invention possess a lower toxicity than prior art Fc-CLEC-1 fusion, thereby reducing risks associated with the administration of the compound in patients in need thereof (e.g. adverse effects, tolerance), and/or allowing the administration of higher doses of Fc-CLEC-1 fusion proteins to enhance its therapeutic effect in vivo. Further, the therapeutic capability of the heterodimeric Fc-CLEC-1 fusion molecules of the present invention is illustrated in cancer models, thereby illustrating that these fusion molecules have a potent therapeutic effect in vivo. It is also provided data illustrating that the heterodimeric Fc-CLEC-1 fusion molecules of the invention are able to bind to a ligand of CLEC-1, referenced CLEC-1-ligand or CLEC-1L, with the same affinity than prior art Fc-CLEC-1 fusion protein or CLEC-1-HIS protein. Further, the heterodimeric Fc-CLEC-1 fusion molecules of the invention are able to bind to tumor cells expressing a ligand of CLEC-1.

The heterodimeric Fc-CLEC-1 fusion molecules of the invention have an enhanced bioavailability in vivo as compared to prior art Fc-CLEC-1 fusion proteins. As illustrated in an example of the invention, in vivo concentration of the heterodimeric Fc-CLEC-1 fusion molecules is detectable during a longer period than prior art Fc-CLEC-1 fusion proteins; while the prior art fusion proteins reach the lower limit of quantification (LLOQ), concentration of the heterodimeric Fc-CLEC-1 fusion molecules of the invention is maintained near its initial concentration.

The heterodimeric Fc-CLEC-1 fusion molecules of the invention do not form aggregate, thereby reducing their immunogenicity. While prior art Fc-CLEC-1 fusion protein may in certain circumstances form aggregates, thereby initiating an immune response, against the aggregates, the heterodimeric Fc-CLEC-1 fusion molecules of the invention do not form aggregate and do not lead to a raise in the secretion of pro-inflammatory cytokines (in particular IL-6) when administered.

In a first aspect, the invention relates to a fusion molecule comprising:
(i) At least one binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising:
   a. a first Fc polypeptide chain comprising a first CH3 domain (CH3B chain or hole chain), and
   b. a second Fc polypeptide chain comprising a second CH3 domain (CH3A chain or knob chain),
   wherein the first and second CH3 domains are different,
   one of the at least one binding moiety being fused to the C-terminal end or the N-terminal end of the first Fc polypeptide, and/or one of the at least one binding moiety being fused to the C-terminal end or N-terminal end of the second Fc polypeptide chain.

In another aspect, the invention related to a fusion molecule comprising:
(i) At least one binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising:
   a. a first Fc polypeptide chain comprising from its N-terminal end to its C-terminal end: a hinge region, a CH2 domain, a first CH3 domain (CH3B chain or hole chain), and a linker, and
   b. a second Fc polypeptide chain comprising a second CH3 domain (CH3A chain or knob chain), in particular comprising from its N-terminal end to its C-terminal end: a hinge region, a CH2 domain and a second CH3 domain (CH3A chain or knob chain),
the at least one binding moiety being fused to the C-terminal end or the N-terminal end of the first Fc polypeptide, in particular the C-terminal end of the first Fc polypeptide.

In another aspect, the invention related to a fusion molecule comprising:
(i) At least one binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising:
   a. a first Fc polypeptide chain comprising a first CH3 domain (CH3B chain or hole chain), in particular comprising from its N-terminal end to its C-terminal end: a hinge region, a CH2 domain and a first CH3 domain (CH3B chain or hole chain) and
   b. a second Fc polypeptide chain comprising from its N-terminal end to its C-terminal end: a hinge region, a CH2 domain, a second CH3 domain (CH3B chain or hole chain), and a linker;
the at least one binding moiety being fused to the C-terminal end or the N-terminal end of the second Fc polypeptide, in particular the C-terminal end of the second Fc polypeptide.

In another aspect, the invention relates to a fusion molecule comprising:
(i) At least one binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising
   a. a first Fc polypeptide chain comprising a first CH3 domain (CH3B chain or hole chain), and
   b. a second Fc polypeptide chain comprising a second CH3 domain (CH3A chain or knob chain),
wherein the first and second CH3 domains are different,
one of the at least one binding moiety being fused to the C-terminal end or the N-terminal end of the first Fc polypeptide, and/or one of the at least one binding moiety being fused to the C-terminal end or N-terminal end of the second Fc polypeptide chain.
   for use as a medicament, particularly in the treatment of a subject, in particular a human subject, who has a disease selected from a cancer, or an infectious disease, or sepsis, an autoimmune disease, or an inflammatory disease, including acute or chronic inflammatory diseases, in particular a cancer listed in the present description, in particular liquid cancers, solid cancers, cancers expressing at least one ligand of human CLEC-1, cancer with CLEC-1-ligand-positive tumors (also referenced cancer with CLEC-1L-positive tumors), cancers with CLEC-1L-positive tumor cells, cancers with glioma cells, breast cancer, hepatocellular carcinoma, lymphoma, more particularly B-cell lymphoma, colon cancer, thyroid cancer, liver cancer, testicular cancer, renal cancer, melanoma, colorectal cancer, nasopharyngeal carcinoma, adenocarcinoma, pancreatic cancer, or selected in the group of a chronic infection, a sepsis, an infection, a viral infection, in particular by a Coxsackievirus or by an encephalitis virus, more particularly by Coxsackievirus B3 or Japanese encephalitis virus, a fungi infection, a cardiovascular disease, an auto-immune disease, in particular Sjogren's syndrome or systemic lupus erythematosus or systemic sclerosis, an inflammatory disease.

In a particular embodiment of the invention, at least a binding moiety of the fusion molecule of the invention comprises or consists of a portion of the extracellular domain of human CLEC-1 comprises at least 50 amino acid residues, in particular at least 100 amino acid residues, more particularly at least 200 amino acid residues, and shares at least 70% identity, in particular at least 80% identity, more particularly at least 90% identity, even more particularly at least 95% identity with the extracellular domain of human CLEC-1 of SEQ ID No. 2.

In a particular embodiment of the invention, the portion of the extracellular domain of human CLEC-1 comprises at least the amino acid sequence set forth in SEQ ID No.

2.

In a particular embodiment of the invention, the CH3 domains of the first Fc polypeptide chain and the second Fc polypeptide are selected from the group of the CH3 domains referenced KiH, KiH_{S-S}, HA-TF, ZW1, 7.8.90, DD-KK, EW-RVT, SEED and A107. In a particular aspect of the invention, it is provided a fusion molecule comprising:
(i) At least one binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising
a. a first Fc polypeptide chain comprising a first CH2 domain (CH2B chain or hole chain), and
b. a second Fc polypeptide chain comprising a second CH2 domain (CH2A chain or knob chain),
wherein the first and second CH2 domains are different,
one of the at least one binding moiety being fused to the C-terminal end or the N-terminal end of the first Fc polypeptide, and/or one of the at least one binding moiety being fused to the C-terminal end or N-terminal end of the second Fc polypeptide chain. According to this embodiment, the fusion molecule may further comprise one (on a single Fc polypeptide chain) or two CH3 domains (each on a single Fc polypeptide chain), the CH3 domains being either different or identical, in particular different. The Fc polypeptide chains may further comprise hinge regions and/or linker regions as disclosed herein.

In a particular embodiment of the invention, a single binding moiety is present within the fusion protein, the binding moiety being fused to either the C-terminal end or the N-terminal end of the first Fc polypeptide chain, in particular the C-terminal end of the first Fc polypeptide chain.

In a particular embodiment of the invention, a binding moiety is fused to the C-terminal end or the N-terminal end of the first Fc polypeptide chain, in particular the C-terminal end of the first Fc polypeptide chain, and wherein a binding molecule, different from the binding moiety associated the first Fc polypeptide chain, is fused to the C-terminal end or the N-terminal end of the second Fc polypeptide chain. The “binding molecule” may be a functional equivalent of a protein (like but not limited to a portion of the extracellular domain of a protein of interest) or an antigen binding molecule. In particular when the at least one binding moiety is present at one end of the same Fc polypeptide chain, the binding molecule is at the opposite end of the Fc polypeptide chain.

In a particular embodiment of the invention, a binding moiety is fused to the C-terminal end or the N-terminal end of the first Fc polypeptide chain, in particular the C-terminal end of the first Fc polypeptide chain, and another binding moiety is fused to the C-terminal end or the N-terminal end of the second Fc polypeptide chain, in particular the C-terminal end of the second Fc polypeptide chain, the binding moieties being identical. In this case, the two binding moieties are functional equivalent of human CLEC-1 and comprises or consists of at least a portion of the extracellular domain of human CLEC-1.

In a particular embodiment of the invention, the at least one binding moiety is fused to the Fc polypeptide chain through a linker peptide, more particularly through a linker peptide of SEQ ID No. 13.

In a particular embodiment of the invention, the first Fc polypeptide chain, or the second Fc polypeptide chain, or both the first and the second Fc polypeptide chains comprise(s) a CH2 domain, in particular at the N-terminal end of the CH3 domain.

In a particular embodiment of the invention, the first Fc polypeptide chain, or the second Fc polypeptide chain, or both the first and the second Fc polypeptide chains comprise(s) a hinge region, in particular at the N-terminal end of the Fc polypeptide chain, more particularly at the N-terminal end of the CH2 domain when present.

In a particular embodiment of the invention, the fusion molecule further comprises:
(iii) At least one antigen-binding domain comprising or consisting of a variable domain of an antibody or antigen-binding fragment thereof, said antigen-binding domain being fused to either the first Fc polypeptide chain or the second Fc polypeptide chain, or a first antigen-binding domain being fused to the first Fc polypeptide chain and a second antigen-binding domain being fused to the second Fc polypeptide chain. In a particular embodiment, the antigen-binding domain binds to an antigen or an epitope expressed by macrophages, and/or lymphocytes, in particular B cell and/or T cell, and/or tumor cells. In a preferred embodiment, the antigen-binding domain binds to an antigen or an epitope selected from the group consisting SIRPalpha, SIRPbeta, SIRPgamma, CD47, CTLA-4, CD86 (B7.2), CD28, CD40, CD40L, ICOS, ICOS-L, OX40L, GITR, HVEM, BTLA, CD160, LIGHT, TNFRSF25, 2B4, CD48, Tim1, Tim3, Tim4, Gal9, LAG-3, CD40, CD40L, CD70, CD27, VISTA, B7H3, B7H4 (B7x), TIGIT, CD112, HHLA2 (B7-H7), TMIGD2 (CD28H), Butyrophilin-like2 (BTNL2), SIGLEC, AXL, B7.1, B7-DC, B7-H1, B7-H2, B7-H3, B7-H4, CD19, CD20, CD22, CD24, CD137 (4-1BB), CD137L (4-1BBL), CEA, CXCR3, CXCR4, EGFR, EGFRVIII, ELTD1, EMR1, EMR2, EMR3, EMR4P, ENG, EPCAM, EPHR, PD-L1, TLR1, TLR10, TLR2, TLR3, TLR4, VEGFR, VEGFR2, VIPR1, VIPR2, CD101, CD3, CD30, CD38, CD39, CD44, DR3, LFA-1, NKG2D, PD-1, and PDL2. In particular when the at least one binding moiety is present at one end of the same Fc polypeptide chain, the antigen-binding domain is at the opposite end of the Fc polypeptide chain.

In a particular embodiment, the antigen-binding domain is fused to the N-terminal end of either the first Fc polypeptide chain or the second Fc polypeptide chain, or wherein the first antigen-binding domain is fused to the N-terminal end of the first Fc polypeptide chain and the second antigen-binding domain is fused to the second Fc polypeptide chain, in particular with the N-terminal end of a CH2 domain or, when present, a hinge region.

In a particular embodiment, two antigen-binding domains are present within the fusion molecule, each being associated with a single Fc polypeptide chain, the first antigen-binding domain and the second antigen-binding domain binding to two different epitopes, in particular to two different antigens, or binding to the same epitope or to the same antigen.

In a particular embodiment of the invention, it is provided a dimer molecule comprising two fusion molecules according to the invention.

In a particular embodiment of the invention, it is provided a soluble fusion molecule according to the invention.

In a particular embodiment of the invention, it is provided a soluble dimer molecule comprising two fusion molecules according to the invention.

In a particular embodiment of the invention, it is provided a bispecific fusion molecule. In a particular embodiment of the invention, it is provided a monospecific fusion molecule.

In a particular embodiment of the invention, it is provided a fusion molecule comprising or consisting of the amino acid sequence set forth in SEQ ID No. 10 and SEQ ID No. 12, or SEQ ID No. 3 and SEQ ID No. 5.

In another aspect, the invention relates to a fusion molecule that is an antagonist of the binding between CLEC-1 and one of its ligands, in particular between human CLEC-1 and human CLEC-1L, for its use as a medicament, in particular for the treatment of a subject, in particular a human subject, who has a disease selected from cancer listed in the present description, in particular cancers with CLEC-1L-positive tumor cells, cancers with glioma cells, breast cancer, hepatocellular carcinoma, lymphoma, more particularly B-cell lymphoma, colon cancer, thyroid cancer, liver cancer, testicular cancer, renal cancer, melanoma, colorectal cancer, adenocarcinoma, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease. Structures of different fusion molecules according to the invention are illustrated on FIG. 1.

### Definitions

As used herein, the terms “CLEC-1” and “CLEC-1A” relates to a CLEC-1A protein from a mammal species, preferably a human CLEC-1 or CLEC-1A. A reference sequence of the human CLEC-1A receptor corresponds to the sequence associated to the Accession number Q8NC01 Uniprot. Preferably, the term “human CLEC-1” refers to the protein of amino acid sequence referenced by the Q8NC01 Uniprot accession number and encoded by CLEC-1 gene referenced by the 51267 NCBI accession number. In the present description, the terms CLEC-1A, CLEC-1, CLECA, CLEC-1, Clec1, Clec-1, CLEC-A1 and Clec-1A are used interchangeably and all designate the CLEC1 receptor of a mammal corresponding to human CLEC-1A receptor characterized by the amino acid sequence associated to the Accession number Q8NC01 Uniprot, an orthologue protein thereof, or a homologous protein thereof.

Preferably, the term “human CLEC-1” refers to the protein of amino acid sequence referenced by the Q8NC01 Uniprot accession number and encoded by CLEC-1 gene referenced by the 51267 NCBI accession number. CLEC-1 may be characterized by the amino acid sequence set forth in SEQ ID No. 1 In a particular embodiment, the amino acid sequence of the extracellular domain of human wild type CLEC-1 comprises or consists in the sequence of

(SEQ ID NO. 2)
QYYQLSNTGQDTISQMEERLGNTSQELQSLQVQNIKLAGSLQHVAEKLC
RELYNKAGAHRCSPCTEQWKWHGDNCYQFYKDSKSWEDCKYFCLSENST
MLKINKQEDLEFAASQSYSEFFYSYWTGLLRPDSGKAWLWMDGTPFTSE
LFHIIIDVTSPRSRDCVAILNGMIFSKDCKELKRCVCERRAGMVKPESL
HVPPETLGEGD.

The fusion molecule is a “fusion protein” that comprises all or part (typically biologically active) of a functional equivalent of CLEC-1 operably linked to at least one heterologous polypeptide (i.e., a polypeptide other than the same polypeptide) that is issued, derived or selected from Fc polypeptides. Within the fusion protein, the term “fused to” is intended to indicate that the functional equivalent of CLEC-1 and the heterologous polypeptide are fused in-frame to each other. The heterologous polypeptide can be fused to the N-terminus or C-terminus of the functional equivalent of CLEC-1 of the present invention. As detailed in the description related to the Fc polypeptides domains, the domains present on the first and second Fc polypeptide chains are not fused, since the first and second Fc polypeptide chains are associated through sub-nanomolar affinity, and by disulfide linkages in the hinge region when present.

According to a particular embodiment of the invention, a fusion molecule is provided that features inter alia a functional equivalent of CLEC-1 through the at least portion of the extracellular domain of CLEC-1. Accordingly, at least a fragment of the extracellular domain of CLEC-1 is fused to a constant domain of a human immunoglobulin or a fragment thereof that comprises at least two Fc polypeptide chains, referenced “heterodimeric Fc polypeptide” or “Fc polypeptide chains” or “Fc fragment”; the combination of the immunoglobulin and the fragment of the extracellular domain of CLEC-1 forms a fusion molecule that can possess many of the valuable chemical and biological properties of human antibodies. The Fc polypeptide chains exhibit a spontaneous pairing interaction between a first Fc polypeptide chain and a second Fc polypeptide chain, the first and the second Fc polypeptides being different at least in their CH3 domain and/or CH2 domain, in particular at least in their CH3 domain, in particular only within their CH3 domain, more particularly in their CH2 and CH3 domains. Thus, the first and second Fc polypeptide chains of the fusion molecule form a heterodimer via their heterodimerization.

In a particular embodiment of the fusion molecule of the invention, the Fc fragment of a human immunoglobulin contained in the Fc-CLEC-1 fusion molecule is the Fc fragment of a human IgG1 or IgG4 and the Fc fragment is fused to the N-terminal end or the C-terminal end of the extracellular domain of the mammalian CLEC-1 domain, in particular of the human CLEC-1 domain.

As used herein, the term “polypeptide” means a polymer of amino acids having every or any length in amino acid residues. Thus, peptides, oligopeptides and proteins are included in the definition of “polypeptide” and these terms are used interchangeably throughout the specification, as well as in the claims. The term “polypeptide” does not exclude post-translational modifications that include but are not limited to phosphorylation, acetylation, glycosylation and the like.

### Heterodimeric Fc Polypeptide Chain or Portion Thereof.

The fragment crystallizable region (Fc region or Fc) is the tail region of an antibody that interacts with cell surface receptors called Fc receptors and some proteins of the complement system. Usually, The Fc region of antibody isotypes is composed of two identical protein fragments, derived from the constant domains of the antibody's two heavy chains (referenced CH2 and CH3, and when present CH4). IgA, IgD and IgG Fc regions contain 2 heavy chain constant domains, while IgM and IgE Fc regions contain three heavy chain constant domains in each polypeptide chain. In the context of IgA, IgD and IgG antibodies, the IgA, IgD and IgG isotypes each have three CH regions, two of them belonging to the Fc region. As an example, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. The Fc region of an IgG consists of two paired CH3 domains and, in contrast, two CH2 domains that are separated and do not interact but have two oligosaccharide chains interposed between them. Wild-type Fc homodimerization is mediated by a large, tightly packed interface between two identical CH3 domains with sub-nanomolar affinity, and subsequently by disulfide linkages in the hinge region. Heterodimeric Fc molecules have been mainly engineered through the replacement of homodimer-favoring interactions at the CH3 domain interface with heterodimer-favoring interactions. This is achieved by introducing asymmetric mutations in each CH3 domain of each Fc chain, which promotes the assembly of Fc chains from two different antibodies.

The functional equivalent of the extracellular domain of CLEC-1 (i.e. at least one binding moiety present within the fusion molecule of the invention) is fused to an immunoglobulin constant domain (Fc region) to form an immunoadhesin. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient and are safe for chronic or repeated use. In some embodiments, the Fc polypeptide is from a native sequence Fc region. In some embodiments, the Fc polypeptide is a variant Fc region. In still another embodiment, the Fc polypeptide is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG4. In some embodiments, the extracellular domain of CLEC-1 and the immunoglobulin sequence portion of the immunoadhesin are linked by a minimal linker. As used herein, the term “linker” refers to a sequence of at least one amino acid that links the polypeptide of the invention and the immunoglobulin sequence portion. Such a linker may be useful to prevent steric hindrances. In some embodiments, the linker has 4; 5; 6; 7; 8; 9; 10; 11; 12; 13; 14; 15; 16; 17; 18; 19; 20; 21; 22; 23; 24; 25; 26; 27; 28; 29; 30 amino acid residues. However, the upper limit is not critical but is chosen for reasons of convenience regarding e.g. biopharmaceutical production of such polypeptides. The linker sequence may be the linker referenced (G4S)3 in the present description. The linker sequence may be a naturally occurring sequence or a non-naturally occurring sequence. If used for therapeutic purposes, the linker is typically non-immunogenic in the subject to which the immunoadhesin is administered.

In the present invention, it is provided a fusion molecule with at least two different protein fragments derived from the constant domains of an antibody's heavy chain. These two different protein fragments are respectively referenced the first Fc polypeptide chain, and the second Fc polypeptide chain. In particular, each of the first and the second Fc polypeptide chains comprises a CH3 domain and/or a CH2 domain, referenced either the first CH3 (or CH2) domain or the second CH3 (or CH2) domain respectively. Particularly, the first CH3 (or CH2) domain and the second CH3 (or CH2) domain are different, i.e. they do not share the same amino acid residue sequences (e.g. they do not have 100% identity). Such CH3 (or CH2) domains may differ by a single amino acid residue, or by 2 amino acid residues, or by 3 amino acid residues or by 4 amino acid residues, or by 5 or more amino acid residues. In a preferred embodiment of the invention, the first CH3 (or CH2) domain and the second CH3 (or CH2) domain are able to interact allowing pairing of the first and second Fc polypeptide chains.

In a particular embodiment of the invention, the two Fc polypeptide chains are issued or derived or selected from the Fc region of a human immunoglobulin heavy chain, in particular an IgG immunoglobulin heavy chain, more particularly from a IgG1, IgG2, IgG3, IgG4 immunoglobulin heavy chain. More particularly, the Fc polypeptide chain is issued, derived or selected from IgG4 Fc-region. More particularly, the Fc polypeptide chain is issued, derived or selected from IgG4 Fc-region with the substitution S228P that allows enhance stability of the Fc region.

In an embodiment of the invention, each Fc polypeptide chain comprises a CH3 domain.

The CH3 domain may be derived, issued or selected from an immunoglobulin heavy chain, in particular a human immunoglobulin heavy chain, more particularly from an IgG heavy chain, for example from IgG1, IgG2, IgG3, IgG4 heavy chains.

In an embodiment of the invention, each Fc polypeptide chain comprises a truncated Fc region or a fragment of a Fc region comprising a CH3 domain.

In an embodiment of the invention, the heterodimeric Fc domain comprises a first Fc chain and a complementary second Fc chain based on the “knobs and holes” technology. As an example, the first Fc chain is a “knob” or K chain, meaning that it comprises the substitution characterizing a knob chain, and the second Fc chain is a “hole” or H chain, meaning that it comprises the substitution characterizing a hole chain. And vice versa, the first Fc chain is a “hole” or H chain, meaning that it comprises the substitution characterizing a hole chain, and the second Fc chain is a “knob” or K chain, meaning that it comprises the substitution characterizing a knob chain. In a preferred aspect, the first Fc chain is a “hole” or H chain and the second Fc chain is a “knob” or K chain.

In a preferred embodiment of the invention, the first Fc polypeptide chain and the second Fc polypeptide chain corresponds to the pairs listed in the following table:

TABLE 1
list of complementary Fc polypeptide chains that can be associated
within the heterodimeric immunoglobulin of the fusion protein.

| Hetorodi- | | |
|---|---|---|
| meric | | Complementary Fc chain |
| reference | Fc chain (“Hole chain”) | (i.e. “Knob chain”) |
| KIH | T366S/L368A/Y407V | T366W |
| KIH_{S-S} | T366S/L368A/Y407V/Y349C | T366W/S354C |
| HA-TF | Y349T/T394F | S364H/F405A |
| ZW1 | T350V/T366L/K392L/T394W | T350V/L351Y/F405A/Y407V |
| 7.8.60 | E345R/Q347R/T366V/K409V | K360D/D399M/Y407A |
| DD-KK | D399K/E356K | K409D/K392D |
| EW-RVT | Q347R/D399V/F405T | K360E/K409W |
| EW- | Q347R/D399V/F405T/S354C | K360E/K409W/Y349C |
| RVT_{S-S} | | |
| A107 | E357N/D399V/F405T | K370E/K409W |
| | D221E/P228E/L368E | D221R/P228R/K409R |
| | C220E/P228E/368E | C220R/E224R/P228R/K409R |
| | S364K/E357Q | L368D/K370S |
| | L368D/K370S | S364K |
| | L368E/K370S | S364K |
| | T411T/E360E/Q362E | D401K |
| | L368D/K370S | S364K/E357L |
| | K370S | S364K/E357Q |
| | F368D/K370S | S364K |
| | F368D/K370S | S364K/E357F |
| | F368D/K370S | S364K/E357Q |
| | T411E/K360E/Q362E | D401K |
| | F368E/K370S | S364K |
| | K370S | S364K/E357Q |
| | T366S/F368A/Y407V | T366W |
| | T366S/L368A/Y407V/Y349C | T366W/S354C |

In a preferred embodiment, the “Hole” Fc polypeptide chain comprises the following substitutions T366S, L368A, Y407V and Y349C. The Hole Fc chain may further comprise additional substitutions.

In a preferred embodiment, the “Knob” Fc polypeptide chain comprises the following substitutions T366W and S354C. The Knob Fc chain may further comprise additional substitutions.

In a preferred embodiment, the “Hole” Fc polypeptide chain comprises the following substitutions T366S, L368A, Y407V and Y349C, and the “Knob” Fc polypeptide chain comprises the following substitutions T366W and S354C. The two Fc chains may further comprise additional substitutions.

In another embodiment, each Fc polypeptide chain comprises a CH3 domain as disclosed here above, and at least a portion CH2 domain, more particularly a full CH2 domain. The CH2 domain may be derived, issued or selected from an immunoglobulin heavy chain, in particular a human immunoglobulin heavy chain, more particularly from an IgG heavy chain, for example from IgG1, IgG2, IgG3, IgG4 heavy chains.

In another embodiment, each Fc polypeptide chain comprises a CH3 domain as disclosed here above and a hinge region. The hinge region is a short sequence of the heavy chains of antibodies linking the Fab (Fragment antigen binding) region to the Fc region. The hinge region may be derived, issued or selected from an immunoglobulin heavy chain, in particular a human immunoglobulin heavy chain, more particularly from an IgG heavy chain, for example from IgG1, IgG2, IgG3, IgG4 heavy chains. One useful group of hinge sequences are derived from the hinge region of heavy chain antibodies as described in WO 96/34103 and WO 94/04678. Other examples are poly-alanine linker sequences. The hinge region may correspond to the amino acid sequence set forth in SEQ ID No. 20.

In another embodiment, each Fc polypeptide chain comprises a constant domain issued or derived form a IgG1 isotype, or a IgG4 isotype.

In an embodiment, the fusion molecule of the invention comprises the amino acid residues set forth in SEQ ID No. 3, which is a KIH-Fc-G1 Knob chain.

In an embodiment, the fusion molecule of the invention comprises the amino acid residues set forth in SEQ ID No. 4, which is a KIH-Fc-G1 Hole chain.

In an embodiment, the fusion molecule of the invention comprises the amino acid residues set forth in SEQ ID No. 3, and the amino acid residues set forth in SEQ ID No. 4.

In an embodiment, the fusion molecule of the invention comprises the amino acid residues set forth in SEQ ID No. 5, which is a KIH-Fc-G1-CLEC-1 Hole chain.

In an embodiment, the fusion molecule of the invention comprises the amino acid residues set forth in SEQ ID No. 5, and the amino acid residues set forth in SEQ ID No. 3.

In an embodiment, the fusion molecule of the invention comprises a Fc polypeptide chain issued or derived from the amino acid residues set forth in SEQ ID No. 6, which is a Fc-G1 chain. Mutations disclosed herein (see table 1) may be present in this chain. In an embodiment, the fusion molecule of the invention comprises a Fc polypeptide chain issued or derived from the amino acid residues set forth in SEQ ID No. 7, which is a Fc-G1e3 chain. Mutations disclosed herein (see table 1) may be present in this chain.

In an embodiment, the fusion molecule of the invention comprises a Fc polypeptide chain issued or derived from the amino acid residues set forth in SEQ ID No. 8, which is a FcG1-N297A chain. Mutations disclosed herein (see table 1) may be present in this chain.

In an embodiment, the fusion molecule of the invention comprises a Fc polypeptide chain issued or derived from the amino acid residues set forth in SEQ ID No. 9, which is a FcG4-S228P chain. Mutations disclosed herein (see table 1) may be present in this chain.

In an embodiment, the fusion molecule of the invention comprises a Fc polypeptide chain issued or derived from the amino acid residues set forth in SEQ ID No. 10, which is a KIH-FcG4 Knob chain.

In an embodiment, the fusion molecule of the invention comprises a Fc polypeptide chain issued or derived from the amino acid residues set forth in SEQ ID No. 11, which is a KIH-FcG4 Hole chain.

In an embodiment, the fusion molecule of the invention comprises a Fc polypeptide chain issued or derived from the amino acid residues set forth in SEQ ID No. 10, and the amino acid residues set forth in SEQ ID No. 11.

In an embodiment, the fusion molecule of the invention comprises a Fc polypeptide chain issued or derived from the amino acid residues set forth in SEQ ID No. 12, which is a KIH-FcG4-CLEC-1 Hole chain.

In an embodiment, the fusion molecule of the invention comprises or consists of a Fc polypeptide chain issued or derived from the amino acid residues set forth in SEQ ID No. 10, and the amino acid residues set forth in SEQ ID No. 12.

In an embodiment, the fusion molecule comprises:
(i) A single binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising
a. a first Fc polypeptide chain comprising a first CH3 domain (CH3B chain or hole chain), and
b. a second Fc polypeptide chain comprising a second CH3 domain (CH3A chain or knob chain),
wherein the first and second CH3 domains are different,
the binding moiety being fused to the C-terminal end of the first Fc polypeptide or the second Fc polypeptide chain,
and
(iii) an antigen-binding domain comprising or consisting of a variable domain of an antibody or antigen-binding fragment thereof, in particular that binds to PD-1, said antigen-binding domain being fused to the N-terminal end of the Fc polypeptide chain fused to the binding moiety.

In a particular embodiment, the binding moiety and the antigen-binding domain are both fused to the Hole Fc polypeptide chain.

### CLEC-1 Binding Moiety

The term “a functionally equivalent fragment” as used herein may mean any fragment or assembly of fragments of CLEC-1. Accordingly, the present invention provides a polypeptide, in particular a functional equivalent, capable of lowering/reducing or inhibiting binding of CLEC-1 to at least one of its ligand, which polypeptide comprises consecutive amino acids having a sequence which is the sequence of at least a portion of an extracellular domain of CLEC-1 for a functional equivalent of CLEC-1, which in a preferred embodiment is a portion that binds to at least one ligand of CLEC-1, in particular to human CLEC-1L.

In a particular embodiment of the invention, the extracellular domain of CLEC-1 is a polypeptide that comprises at least 20, in particular at least 25, in particular at least 30, in particular at least 40, in particular at least 50 amino acid residues, in particular at least 80 amino acid residues, in particular at least 100 amino acid residues, in particular at least 200 amino acid residues, in particular at least 300 contiguous amino acid residues within sequence SEQ ID No. 2. Alternatively, the extracellular domain of CLEC-1 is a peptide or a polypeptide that comprises at least 20 amino acid residues, in particular at least 25 amino acid residues, in particular at least 30 amino acid residues, in particular at least 40 amino acid residues, in particular at least 50 amino acid residues, in particular at least 80 amino acid residues, in particular at least 100 amino acid residues, in particular at least 150 amino acid residues, in particular at least 180 amino acid residues, in particular at least 200 amino acid residues, in particular at least 210 and has at least 70% identity, in particular at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity, or share 100% identity, with the extracellular domain of CLEC-1 is of SEQ ID No. 2. The extracellular domain of CLEC-1 may be a polypeptide fused to a linker sequence, which comprises of consists of an amino acid sequence having at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% or share 100% identity, with the extracellular domain of CLEC-1 of SEQ ID No. 2.

The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”.

As used herein, a “functional equivalent” of CLEC-1 is a compound which is capable of binding to at least one CLEC-1 ligand, in particular to CLEC-1L, thereby preventing its interaction with CLEC-1. The term “functional equivalent” includes fragments, mutants, and muteins of CLEC-1. The term “functionally equivalent” thus includes any equivalent of CLEC-1 obtained by altering the amino acid sequence, for example by one or more amino acid deletions, substitutions or additions such that the protein analogue retains the ability to bind to its ligand. Amino acid substitutions may be made, for example, by point mutation of the DNA encoding the amino acid sequence.

Functional equivalents of CLEC-1 include but are not limited to molecules that bind to at least one ligand of CLEC-1, in particular to CLEC-1L, and comprise all or a portion of the extracellular domain of CLEC-1 so as to form a molecule that is capable to bind to at least one ligand of CLEC-1, in particular to CLEC-1L (in particular so as to form a soluble receptor that is capable to trap at least one ligand of CLEC-1). The functional equivalents include soluble forms of CLEC-1. A suitable soluble form of these proteins, or functional equivalents thereof, might comprise, for example, a mutated, in particular truncated form of the protein from which the transmembrane domain has been removed by chemical, proteolytic or recombinant methods. Particularly, the functional equivalent consists of an amino acid sequence having at least 70%, in particular at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity with the corresponding protein over the entire length of the corresponding protein. As used herein, the term “corresponding protein” refers to the protein for which the functional equivalent of the invention has similar function. The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”.

In an embodiment of the invention, the portion of the extracellular domain of human CLEC-1 correspond to a truncated portion of the extracellular domain of CLEC-1, wherein the N-terminal end of the extracellular domain of CLEC-1, in particular of SEQ ID No. 2, has been deleted. The N-terminal end may correspond to the first 10, or first 11, or first 13, or first 14, or first 15, or first 16, or first 17, or first 18, or first 19, or first 20, or first 21, or first 22, or first 23, or first 24, or first 25 amino acid residues of SEQ ID No. 2. Alternatively, the N-terminal end of the extracellular domain of CLEC-1 may correspond to at least the first amino acid residue localized at the N-terminal end of the extracellular domain of CLEC-1 to at most 10% of the amino acid residues of the extracellular domain of CLEC-1 localized at the N-terminal end. In a preferred embodiment, the truncated portion of the extracellular domain of CLEC-1 may have the sequence of amino acid residues set forth in SEQ ID No. 19.

### Linker Peptide and Hinge Domain

In a particular embodiment of the invention, any binding moiety comprised within the fusion molecule may be fused to a Fc polypeptide chain through a linker peptide. A linker peptide usually ensures that the attached binding moiety connects with the Fc polypeptide chain in such a conformation for each to exert their functions.

In a particular embodiment, each binding moiety is preferably linked to the Fc polypeptide chain through a linker peptide. In a particular embodiment, the binding moiety may be fused to the Fc polypeptide chain through a linker peptide when the binding moiety is fused toward the C-terminal end of the Fc polypeptide chain. As used herein, the term “linker peptide” refers to a sequence of at least one amino acid residue. The linker is usually 1-44 amino acid residues in length. Preferably, the linker has 3-30 amino acid residues. In a particular embodiment, a linker peptide has 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

The linker peptide may be a naturally occurring sequence or a non-naturally occurring sequence. In a particular embodiment, the linker peptide is non-immunogenic in the subject to which the fusion molecule is to be administered. Other examples are poly-alanine linker sequences. Further preferred examples of linker sequences are Gly/Ser linkers of different length including (Gly4Ser)4, (Gly4Ser)3, (Gly4Ser)2, Gly4Ser, Gly3Ser, Gly3, Gly2ser and (Gly3Ser2)3, in particular (Gly4Ser)3. Preferably, the linker is selected from the group consisting of (Gly4Ser)4, (Gly4Ser)3, and (Gly3Ser2)3. Even more preferably, the linker is (GGGGS)3. In a particular embodiment, the linker peptide comprised in the fusion molecule is selected in the group consisting of (Gly4Ser)4 (SEQ ID No. 14), (Gly4Ser)3 (SEQ ID No. 13), (Gly4Ser)2 (SEQ ID No. 15), Gly4Ser (SEQ ID No. 16), Gly3Ser (SEQ ID No. 17), Gly3, Gly2ser and (Gly3Ser2)3 (SEQ ID No. 18), preferably is (Gly4Ser)3. Preferably, the linker is selected from the group consisting of (Gly4Ser)4, (Gly4Ser)3, and (Gly3Ser2)3. In a particular embodiment, the linker peptide is (Gly4Ser)3 of SEQ ID No. 13. In a particular embodiment, the binding moiety can be fused to the Fc chain by a hinge sequence (usually referenced as “hinge”) naturally found in the heavy chain of immunoglobulins for connecting VH domains, in particular for connecting the CH1 domain to the CH2 domain. In a particular embodiment, the binding moiety may be fused to the Fc polypeptide chain through a hinge domain when the binding moiety is fused toward the N-terminal end of the Fc polypeptide chain. One useful group of hinge domains are derived from the hinge region of heavy chain antibodies as described in WO 96/34103 and WO 94/04678.

In an embodiment of the invention, the fusion molecule comprises at least one binding moiety, in particular a single binding moiety, fused to the C-terminal end of the first Fc polypeptide chain. In a preferred embodiment, the at least one binding moiety, or the single binding moiety, is fused to the C-terminal end of the first Fc polypeptide through a linker peptide, in particular a linker peptide comprising or consisting of SEQ ID No. 13. In a particular embodiment, the binding moiety is mutated extracellular domain of CLEC-1, in particular a truncated portion of the extracellular domain of CLEC-1, more particularly having the sequence of amino acid residues set forth in SEQ ID No. 19. As illustrated in the examples of the invention, such a construct is less likely to aggregate.

In an embodiment of the invention, the fusion molecule comprises at least one binding moiety, in particular a single binding moiety, fused to the C-terminal end of the second Fc polypeptide chain. In a preferred embodiment, the at least one binding moiety, or the single binding moiety, is fused to the C-terminal end of the first Fc polypeptide through a linker peptide, in particular a linker peptide comprising or consisting of SEQ ID No. 13. In a particular embodiment, the binding moiety is a mutated extracellular domain of CLEC-1, in particular truncated portion of the extracellular domain of CLEC-1, more particularly having the sequence of amino acid residues set forth in SEQ ID No. 19. As illustrated in the examples of the invention, such a construct is less likely to aggregate.

In an embodiment of the invention, the fusion molecule comprises a heterodimeric immunoglobulin comprising or consisting of a Fc KIH region, a linker region, and a CLEc-1 domain region. In a preferred embodiment, the fusion molecule comprises a heterodimeric immunoglobulin comprising or consisting of a Fc KIH region selected from the left-hand column of table 2, a linker region selected from the center column of table 2, and a CLEC-1 domain selected from the right-hand column of table 2.

TABLE 2
Sequences of various Fc KIH regions, linkers and CLEc-1 domains
that can be present within a heterodimeric immunoglobulin
present in a fusion molecule according to the invention.

| Fc KIH region | Linker | CLEC-1 domain |
|---|---|---|
| SEQ ID No. 3 or | SEQ ID No. 13 or | SEQ ID No. 2 or |
| SEQ ID No. 4 or | SEQ ID No. 14 or | SEQ ID No. 19 |
| SEQ ID No. 10 or | SEQ ID No. 15 or | |
| SEQ ID No. 11 or | SEQ ID No. 16 or | |
| SEQ ID No. 20 + SEQ ID No. | SEQ ID No. 17 or | |
| 21 + SEQ ID No. 22 | SEQ ID No. 18 or | |
| | Gly3 or Gly2ser | |

SEQ ID No. 3 corresponds to the Fc-KIH (Knob) illustrated in the examples of the invention. It is in particular a IgG1 domain with T366W and S354C).

SEQ ID No. 4 corresponds to the Fc-KIH (Hole) illustrated in the examples of the invention. It is in particular a IgG1 domain with T366S, L368A, Y407V and Y349C).

SEQ ID No. 10 corresponds to a Fc-KIH (Hole). It is in particular a IgG4 domain with S228P, T366W and S354C.

SEQ ID No. 11 corresponds to a Fc-KIH (Knob). It is in particular a IgG4 domain with S228P, T366S, L368A, Y407V and Y349C.

SEQ ID No. 20 corresponds to a hinge domain, for example found in the Fc-KIH (Knob or Hole) illustrated in the examples of the invention.

SEQ ID No. 21 corresponds to a CH2 domain, for example found in the Fc-KIH (Knob or Hole) illustrated in the examples of the invention.

SEQ ID No. 22 corresponds to a CH3 domain, for example found in the Fc-KIH (Hole) illustrated in the examples of the invention.

The linkers are those detailed here above.

CLEC-1 domain of SEQ ID No. 2 corresponds to the extracellular domain of CLEC-1. CLEC-1 domain of SEQ ID No. 19 corresponds to a truncated extracellular domain of CLEC-1 as detailed above.

In an embodiment of the invention, the fusion molecule comprises a heterodimeric immunoglobulin comprising a second Fc polypeptide chain comprising or consisting of the following domains:
A Fc-KIH domain of SEQ ID No. 3; and
A linker of SEQ ID No. 13; and
A CLEC-1 domain of SEQ ID NO. 2; and optionally in a preferred embodiment, the first Fc polypeptide chain comprises or consists of the Fc KIH domain of SEQ ID No. 4.

In an embodiment of the invention, the fusion molecule comprises a heterodimeric immunoglobulin comprising a second Fc polypeptide chain comprising or consisting of the following domains:
A Fc-KIH domain of SEQ ID No. 3; and
A linker of SEQ ID No. 13; and
A CLEC-1 domain of SEQ ID NO. 19; and optionally in a preferred embodiment, the first Fc polypeptide chain comprises or consists of the Fc KIH domain of SEQ ID No. 4.

In an embodiment of the invention, the fusion molecule comprises a heterodimeric immunoglobulin comprising a first Fc polypeptide chain comprising or consisting of the following domains:
A Fc-KIH domain of SEQ ID No. 4; and
A linker of SEQ ID No. 13; and
A CLEC-1 domain of SEQ ID NO. 2, and optionally in a preferred embodiment, the second Fc polypeptide chain comprises or consists of the Fc KIH domain of SEQ ID No. 3.

In an embodiment of the invention, the fusion molecule comprises a heterodimeric immunoglobulin comprising a first Fc polypeptide chain comprising or consisting of the following domains:
A Fc-KIH domain of SEQ ID No. 4; and
A linker of SEQ ID No. 13; and
A CLEC-1 domain of SEQ ID NO. 19; optionally in a preferred embodiment, the second Fc polypeptide chain comprises or consists of the Fc KIH domain of SEQ ID No. 3.

In an embodiment of the invention, the fusion molecule comprises a heterodimeric immunoglobulin comprising a first Fc polypeptide chain comprising or consisting of the following domains:
A hinge domain of SEQ ID No. 20; and
A CH2 domain of SEQ ID No. 21; and
A CH3 domain of SEQ ID No. 22; and
A linker of SEQ ID No. 13; and
A CLEC-1 domain of SEQ ID NO. 2; and optionally in a preferred embodiment, the second Fc polypeptide chain comprises or consists of the Fc KIH domain of SEQ ID No. 3.

In an embodiment of the invention, the fusion molecule comprises a heterodimeric immunoglobulin comprising a first Fc polypeptide chain comprising or consisting of the following domains:
A hinge domain of SEQ ID No. 20; and
A CH2 domain of SEQ ID No. 21; and
A CH3 domain of SEQ ID No. 22; and
A linker of SEQ ID No. 13; and
A CLEC-1 domain of SEQ ID NO. 19; and optionally in a preferred embodiment, the second Fc polypeptide chain comprises or consists of the Fc KIH domain of SEQ ID No. 3.

In an embodiment of the invention, the fusion protein comprises a first Fc polypeptide chain comprising of consisting of the amino acid sequence set forth in SEQ ID No. 4 and a second Fc polypeptide chain comprising of the amino acid sequence set forth in SEQ ID No. 3, and at least binding moiety comprising or consisting of the amino acid sequence set forth in Seq ID No. 2. In particular, the at least one binding moiety is linked to the C-terminal end of SEQ ID No. 4 or SEQ ID No. 3 in particular through the linker of SEQ ID No. 13.

In an embodiment of the invention, the fusion protein comprises a first Fc polypeptide chain comprising of consisting of the amino acid sequence set forth in SEQ ID No. 4 and a second Fc polypeptide chain comprising of the amino acid sequence set forth in SEQ ID No. 3, and at least binding moiety comprising or consisting of the amino acid sequence set forth in Seq ID No. 19. In particular, the at least one binding moiety is linked to the C-terminal end of SEQ ID No. 4 or SEQ ID No. 3, in particular through the linker of SEQ ID No. 13.

In an embodiment of the invention, the fusion protein comprises a first Fc polypeptide chain comprising of consisting of the amino acid sequence set forth in SEQ ID No. 5 and a second Fc polypeptide chain comprising of the amino acid sequence set forth in SEQ ID No. 3, the binding moiety of SEQ ID No. 2 being present within the first Fc polypeptide chain.

In an embodiment of the invention, the fusion protein comprises a first Fc polypeptide chain comprising of consisting of the amino acid sequence set forth in SEQ ID No. 4 and a second Fc polypeptide chain comprising of the amino acid sequence set forth in SEQ ID No. 23, the binding moiety of SEQ ID No. 23 being present within the second Fc polypeptide chain.

Other Binding Molecules (Antigen-Binding Domains and Other Binding Molecules Different from the at Least One Binding Moiety of the Fusion Protein)

In a particular embodiment of the invention, the fusion molecule further comprises at least an antigen-binding domain or another binding moiety which is different from the at least one binding moiety comprising or consisting of at least a portion of the extracellular domain of human C-type lectin domain family 1 member A.

The fusion molecule may comprise a further binding moiety which is different from the at least one binding moiety comprising or consisting of at least a portion of the extracellular domain of human CLEC-1, but which is not an antigen-binding domain issued from an antibody or related compound. Such a binding moiety may correspond to the extracellular domain of a protein or a portion thereof, in particular of protein known to be involved in immune response, like but not limited to PD1, PDL1, CTLA4, 4-1BBL, CD80, CD137. The binding moiety should possess the binding capability of its naïve protein for at least one of its ligands.

When the fusion molecule comprises an antigen-binding domain, this domain comprises or consists of a variable domain of an antibody or of an antigen-binding fragment thereof, said domain being fused to either the first Fc polypeptide chain or the second Fc polypeptide chain, or a first antigen-binding domain being fused to the first Fc polypeptide chain and a second antigen-binding domain being fused to the second Fc polypeptide chain.

In a particular embodiment, when an antigen-binding domain is present on a Fc polypeptide chain that is associated with a binding moiety at one end, the antigen-binding fragment or the binding molecule is associated at the opposite end of the Fc polypeptide chain. As an example, when the binging moiety is associated to the C-terminal end of the Fc polypeptide chain, then the antigen-binding domain or binding molecule is associated to the N-terminal end of the Fc polypeptide chain. When the binging moiety is associated to the N-terminal end of the Fc polypeptide chain, then the antigen-binding domain or binding molecule is associated to the C-terminal end of the Fc polypeptide chain.

As used herein, an antigen-binding domain means a part or a fragment of an antibody, i.e. a molecule corresponding to a portion of the structure of an antibody, that exhibits antigen-binding capability for their target, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragments that have a reduced antigen-binding affinity with respect to corresponding 4-chain antibodies are also encompassed for use within the invention. The antigen-binding capability can be determined by measuring the affinity between the antibody and the target fragment of antibody. These antigen-binding fragments may also be designated as “functional fragments” of antibodies.

Antigen-binding fragments of antibodies are fragments which comprise the hypervariable domains designated CDRs (Complementary Determining Regions) of the antibody of reference or part(s) thereof encompassing the recognition site for the antigen. Antigen binding fragments of an antibody that contain the variable domains comprising the CDRs of said antibody encompass Fv, dsFv, scFv, Fab, Fab′, F(ab′)2. In a particular embodiment of the invention, the antigen-binding domain is a Fab domain.

These basic antigen-binding fragments for use according to the invention can be combined together to obtain multivalent antigen-binding fragments, such as diabodies, tribodies or tetrabodies. These multivalent antigen-binding fragments are also part of the present invention when a Fab domain is associated to the fusion molecule. Antigen-binding antibody mimetics are organic compounds that specifically bind antigens, but that are not structurally related to antibodies. They are usually artificial peptides or small proteins with a molar mass of about 3 to 20 kDa. Nucleic acids and small molecules are sometimes considered antibody mimetics as well, but not artificial antibodies, antibody fragments and fusion proteins composed from these. Common advantages over antibodies are better solubility, tissue penetration, stability towards heat and enzymes, and comparatively low production costs. Antibody mimetics are being developed as therapeutic and diagnostic agents. Antigen-binding antibody mimetics may also be selected among the group comprising affibodies, affilins, aptamers, anticalins, affimers, affitins, DARPins, and Monobodies.

In a particular embodiment of the invention, the fusion molecule comprises two antigen-binding domains, each being associated with a single Fc polypeptide chain, the first antigen-binding domain and the second antigen-binding domain binding to the same epitope, or the same antigen, or two different epitopes, or to two different antigens. An epitope is a portion of an antigen molecule to which an antibody binds itself. Thus, when the fusion molecule comprises two antigen-binding domains, the two antigen-binding domains may bind to the same epitope or may bind to two different epitopes localized within the same antigen, or may bind to two different epitopes localized within two different antigens. In other words, when the fusion molecule comprises two antigen-binding domains, they may bind to the same antigen or to two different antigens.

In a particular embodiment of the invention, at least one antigen-binding domain specifically binds to an antigen expressed by macrophages, and/or lymphocytes, in particular B cell or T cell, and/or tumor cells.

In a particular embodiment of the invention, at least one antigen-binding domain specifically binds to an antigen selected from the list consisting of SIRPalpha, SIRPbeta, SIRPgamma, CD47, CTLA-4, CD86 (B7.2), CD28, CD40, CD40L, ICOS, ICOS-L, OX40L, GITR, HVEM, BTLA, CD160, LIGHT, TNFRSF25, 2B4, CD48, Tim1, Tim3, Tim4, Gal9, LAG-3, CD40, CD40L, CD70, CD27, VISTA, B7H3, B7H4 (B7x), TIGIT, CD112, HHLA2 (B7-H7), TMIGD2 (CD28H), Butyrophilin-like2 (BTNL2), SIGLEC, AXL, B7.1, B7-DC, B7-H1, B7-H2, B7-H3, B7-H4, CD19, CD20, CD22, CD24, CD137 (4-1BB), CD137L (4-1BBL), CEA, CXCR3, CXCR4, EGFR, EGFRvIII, ELTD1, EMR1, EMR2, EMR3, EMR4P, ENG, EPCAM, EPHR, PD-L1, TLR1, TLR10, TLR2, TLR3, TLR4, VEGFR, VEGFR2, VIPR1, VIPR2 CD101, CD3, CD30, CD38, CD39, CD44, DR3, LFA-1, NKG2D, PD-1, and PDL2.

In a particular embodiment of the invention, the fusion molecule comprises two antigen-binding domains, each being associated with a single Fc polypeptide chain, both antigen-binding domains binding to the same antigen, selected from the list consisting of SIRPalpha, SIRPbeta, SIRPgamma, CD47, CTLA-4, CD86 (B7.2), CD28, CD40, CD40L, ICOS, ICOS-L, OX40L, GITR, HVEM, BTLA, CD160, LIGHT, TNFRSF25, 2B4, CD48, Tim1, Tim3, Tim4, Gal9, LAG-3, CD40, CD40L, CD70, CD27, VISTA, B7H3, B7H4 (B7x), TIGIT, CD112, HHLA2 (B7-H7), TMIGD2 (CD28H), Butyrophilin-like2 (BTNL2), SIGLEC, AXL, B7.1, B7-DC, B7-H1, B7-H2, B7-H3, B7-H4, CD19, CD20, CD22, CD24, CD137 (4-1BB), CD137L (4-1BBL), CEA, CXCR3, CXCR4, EGFR, EGFRvIII, ELTD1, EMR1, EMR2, EMR3, EMR4P, ENG, EPCAM, EPHR, PD-L1, TLR1, TLR10, TLR2, TLR3, TLR4, VEGFR, VEGFR2, VIPR1, VIPR2, CD101, CD3, CD30, CD38, CD39, CD44, DR3, LFA-1, NKG2D, PD-1, and PDL2.

In a particular embodiment of the invention, the fusion molecule comprises two antigen-binding domains, each binding an antigen, the two antigens recognized by each of the antigen-binding domains being independently selected from the list consisting of SIRPalpha, SIRPbeta, SIRPgamma, CD47, CTLA-4, CD86 (B7.2), CD28, CD40, CD40L, ICOS, ICOS-L, OX40L, GITR, HVEM, BTLA, CD160, LIGHT, TNFRSF25, 2B4, CD48, Tim1, Tim3, Tim4, Gal9, LAG-3, CD40, CD40L, CD70, CD27, VISTA, B7H3, B7H4 (B7x), TIGIT, CD112, HHLA2 (B7-H7), TMIGD2 (CD28H), Butyrophilin-like2 (BTNL2), SIGLEC, AXL, B7.1, B7-DC, B7-H1, B7-H2, B7-H3, B7-H4, CD19, CD20, CD22, CD24, CD137 (4-1BB), CD137L (4-1BBL), CEA, CXCR3, CXCR4, EGFR, EGFRvIII, ELTD1, EMR1, EMR2, EMR3, EMR4P, ENG, EPCAM, EPHR, PD-L1, TLR1, TLR10, TLR2, TLR3, TLR4, VEGFR, VEGFR2, VIPR1, VIPR2, CD101, CD3, CD30, CD38, CD39, CD44, DR3, LFA-1, NKG2D, PD-1, and PDL2.

In particular embodiment, the antigen-binding domain is at the N-terminal end of either the first Fc polypeptide chain or the second Fc polypeptide chain.

In particular embodiment, the antigen-binding domain is at the N-terminal end of the first Fc polypeptide chain and the second antigen-binding domain is fused to the second Fc polypeptide chain.

In particular embodiment, the antigen-binding domain is at the N-terminal end of a CH2 domain or, when present, a hinge region.

### Use of the Fusion Molecule

The fusion molecule is provided for use as a medicament. In particular, the fusion molecule is provided for use in the treatment of a disease in a subject (i.e. a patient, in particular a human patient), in particular a subject having a cancer, or an infectious disease, or sepsis, or an autoimmune disease, or an inflammatory disease, including acute or chronic inflammatory diseases. In another embodiment, the fusion molecule may be provided for use in the prevention of a disease in a subject. As used herein, “treatment” or “treating” is an approach for obtaining beneficial or desired results. Beneficial or desired clinical results include, but are not limited to, one or more of the following: alleviating, preventing or abolishing one or more symptoms resulting from the disease, curing the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. According to an embodiment, the term “treatment” relates to the prophylactic treatment. As used herein, the term “prevent” refers to the reduction in the risk of acquiring or developing a given condition.

In a particular embodiment of the invention, the fusion molecule is provided for use in the treatment of a patient, wherein the modulation of the phagocytosis of target cells, in particular such as cells expressing at least one ligand of CLEC-1, particularly CLEC-1L, more particularly of CLEC-1L-positive tumor cells, by myeloid cells, in particular by dendritic cells and/or macrophages, to improve the outcome of a disease. Thus, in a particular embodiment, it is provided a fusion molecule to increase the phagocytosis of cells, in particular of CLEC1 ligand-positive cells, particularly CLEC-1L-positive cells, more particularly of tumor cells and/or secondary necrotic cells, even more particularly of CLEC-1L-positive tumor cells and/or CLEC-1L-positive secondary necrotic cells, by myeloid cells, particularly by dendritic cells and/or macrophages. Thus, CLEC-1L-positive tumors (i.e. tumors that include at least one cell expressing, in particular abnormally expressing, CLEC-1L) can be targeted by the fusion molecule of the invention. Indeed, it has been shown that antagonists of CLEC-1L-CLEC1 signaling pathway (e.g. compounds binding to CLEC-1Lor CLEC1 that inhibits the binding between CLEC-1 and CLEC-1L, or functional equivalent of CLEC-1L or CLEC1, or compounds that reduce the expression of functional CLEC-1L or of CLEC1, or compounds that reduce or inhibit the signaling pathway induced by the binding between CLEC-1L and CLEC-1) are able to modulate, in particular to enhance, the phagocytosis of target cells, such as tumor cells expressing CLEC-1Lby myeloid cells, in particular by dendritic cells and/or macrophages, thereby leading to their use in therapy or treating disease wherein modulating the phagocytosis of target cells such as tumor cells or cells expressing CLEC-1Lby myeloid cells, in particular dendritic cells and/or macrophages, improves the health of the patient. Thereby, the use of the fusion molecule of the invention, which at least reduces the interaction of CLEC-1 (expressed on the cell surface of dendritic cells and macrophages) to its ligand CLEC-1L (expressed for example at least by tumor cells), could be useful for modulating the phagocytosis of cells such as tumor cells by myeloid cells, in particular dendritic cells and/or macrophages. When CLEC-1A-expressing myeloid cells, in particular macrophages or dendritic cells, interact with cells expressing CLEC-1L, the phagocytosis by these macrophages or dendritic cells may be inhibited or reduced. As an example, it has been shown that Tumor cells (i.e. Raji cells) that express CLEC-1 Lescape phagocytosis exerted by macrophages. By using an antagonist compound according to the invention that modulates the interaction between CLEC-1-expressing myeloid cells and CLEC-1L-expressing cells, in particular CLEC-1L-positive tumor cells, the reduction or inhibition of the phagocytosis of CLEC-1L-expressing cells by myeloid cells, in particular dendritic cells or macrophages, is lowered. Consequently, the phagocytosis of CLEC-1L-expressing cells, like tumor cells, is enhanced in presence of a fusion molecule of the invention.

More particularly, the present invention concerns the use of the fusion molecule, in the treatment of a condition or a disease in a patient wherein said condition or disease is, or is related to, a cancer listed in the present description, in particular cancer with CLEC1 ligand-positive tumor cells, particularly CLEC-1L-positive tumor cells, cancers with glioma cells, breast cancer, hepatocellular carcinoma, lymphoma, more particularly B-cell lymphoma, colon cancer, thyroid cancer, liver cancer, testicular cancer, renal cancer, melanoma, colorectal cancer, adenocarcinoma, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease.

The terms “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to liquid cancers, solid cancers, cancers expressing at least one ligand of human CLEC-1, cancer with CLEC-1-ligand-positive tumors, cancer with CLEC-1L-positive tumor cells, cancer with CLEC-1L-positive tumor, cancer from the bladder, blood, bone, bone marrow, brain, breast, colon, oesophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukaemia; lymphoid leukaemia; plasma cell leukaemia; erythroleukemia; lymphosarcoma cell leukaemia; myeloid leukaemia; basophilic leukaemia; eosinophilic leukaemia; monocyticleukaemia; mast cell leukaemia; megakaryoblasticleukaemia; myeloid sarcoma; and hairy cell leukaemia.

In a particular embodiment, the subject suffers from a cancer selected from the group consisting of bile duct cancer, bladder cancer, bone cancer, brain and central nervous system cancer, breast cancer, Castleman disease, cervical cancer, colorectal cancer, adenocarcinoma, endometrial cancer, oesophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

CLEC-1A is also known to be involved in various pathological conditions including sepsis, infection, allergic inflammation. Tone et al. (Frontiers in Immunology, 2021), Gao et al. (iSciences, 2020), or Zhu z et al. (Invest. Ophthalmo. Vis. Scis., 2021) highlight that blocking CLEC-1A signaling is associated with a modulation of the immune response (CLEC-1 being an immune suppressor of myeloid cells), which could enhance the outcomes of patients.

The present invention also concerns the use of heterodimeric fusion molecule in the treatment of an infectious disease.

The present invention also concerns the use of heterodimeric fusion molecule in the treatment of a sepsis.

The present invention also concerns the use of heterodimeric fusion molecule in the treatment of an autoimmune disease.

The present invention also concerns the use of heterodimeric fusion molecule in the treatment of an inflammatory disease, in particular acute or chronic inflammatory diseases.

The present invention also concerns the use of heterodimeric fusion molecule in the treatment, including the preventive treatment, of a deleterious condition or a disease, in particular wherein the dendritic cells and/or the T cells are involved, and wherein the proliferation of T cells and/or the stimulation of the phagocytosis by myeloid cells, in particular by dendritic cells and/or macrophages, may improve or treat the condition or the disease. In a particular embodiment, the disease or condition is selected from the group consisting of cancer, in particular a cancer as listed here above, more particularly cancers with CLEC-1L-positive tumor cells, liquid cancers, solid cancers, lymphoma, colorectal cancers, adenocarcinoma, mesothelioma or hepatocarcinoma.

### Pharmaceutical Compositions

The invention also concerns a pharmaceutical composition for use in the treatment of a patient having a disease, comprising as a therapeutic agent a fusion molecule as defined herein, either alone or in combination with a second therapeutic agent, with a pharmaceutical suitable vehicle, which are pharmaceutically acceptable for a formulation capable of being administered to a patient in need thereof. These formulations may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

### Nucleic Acid Molecules

The invention also concerns nucleic acid molecule(s) or a combination of nucleic acid molecules encoding at least a portion of the fusion proteins described herein.

To this end, the invention also relates to nucleic acid molecule(s) or combination(s) of nucleic acid molecules encoding a fusion protein according to any one of the definitions disclosed herein. In particular, the nucleic acid molecule(s) encode(s) at least the first Fc polypeptide chain, and/or the second Fc polypeptide chain, and/or the at least one binding moiety disclosed herein. Preferably, the nucleic acid molecule(s) encode(s) the first Fc polypeptide chain, the second Fc polypeptide chain, and the at least one binding moiety. In particular, the nucleic acid molecule(s) may encode(s) a hinge portion, a linker, and/or a CH2 when these elements are present.

The invention may also relate to a combination of a first nucleic acid molecule and a second nucleic acid molecule, wherein a first nucleic acid molecule encodes at least the first Fc polypeptide chain, and wherein a second nucleic acid molecule encodes at least the second Fc polypeptide chain.

Any nucleic acid molecules according to the invention may be inserted within an expression vector, like a plasmid for example, suitable for expression of the encoded sequence within a host cell.

### Combinations of Compounds

The invention also concerns a combination of compounds comprising a first therapeutic agent and at least one further therapeutic agent.

A combination of compounds is a formulation comprising at least two different products, or agents, or compounds, which may be packed together or packed separately, and that are prepared for a simultaneous administration, a co-administration, or a coordinated administration (or sequential administration). In particular, the amount of each compound within the combination may be comprised between 1 μg/kg and 100 mg/kg of weight of the patient.

The first therapeutic agent is a fusion molecule as defined in any embodiment of the invention disclosed herein (i.e., a molecule comprising or consisting of a binding moiety comprising or consisting of at least a portion of the extracellular domain of CLEC-1, and a heterodimeric Fc region).

At least one further therapeutic agent is selected from the list consisting of a tumor-targeting antibody or antigen-binding fragment thereof, in particular a tumor-targeting monoclonal antibody or antigen-binding fragment thereof, more particularly a tumor-targeting monoclonal antibody or antigen-binding fragment thereof which activates and/or enhances the phagocytosis of tumor cells or cells expressing CLEC-1Lby myeloid cells, in particular by dendritic cells and/or macrophages, and still more particularly a monoclonal antibody selected from the group consisting of alemtuzumab, atezolizumab, bevacizumab, cetuximab, herceptin, panitumumab, rituximab, trastuzumab, an anti-PDL-1 antibody, and an anti-CD47 antibody, or another antibody or monoclonal antibody selected from the group consisting of an anti-PD1 antibody and an anti-SIRPa antibody; and/or a chemotherapeutic agent, in particular a cytotoxic agent with anti-proliferative, pro-apoptotic, cell cycle arresting and/or differentiation inducing effect, more particularly a cytotoxic agent selected from the group consisting of cytotoxic antibody, alkylating drugs, anthracyclines, antimetabolites, anti-microtubule agents, topoisomerase inhibitors, alkaloids, bleomycin, antineoplastic drugs, cyclophosphamide. A tumor-targeting antibody may be defined as a therapeutic monoclonal antibody that recognizes tumor-specific membrane proteins, blocks cell signalling, and induces tumor killing through Fc-driven innate immune responses. The chemotherapeutic agent may be a conventional cytotoxic agent, i.e. a compound that induces irreversible lethal lesions through interference with DNA replication, mitosis, etc. following exposure. These agents may have anti-proliferative, pro-apoptotic, cell cycle arresting, and differentiation inducing effects. These agents are preferentially selected from the group consisting of alkylating drugs (cisplatin, chlorambucil, procarbazine, carmustine), anthracyclines and other cytotoxic antibiotics, antimetabolites (i.e. methotrexate, cytarabine, gemcitabine), anti-microtubule agents (i.e. vinblastine, paclitaxel, docetaxel), topoisomerase inhibitors (i.e. etoposide, doxorubicin), alkaloids (i.e. Vincristine, Vinblastine, Vinorelbine, Camptothecin) or bleomycin (inhibiting incorporation of thymidine into DNA strands). The combination may comprise more than one second therapeutic agent selected from the list. The combination may also further comprise additional therapeutic agents, not recited in the list, and/or component(s), like but not limited to pharmaceutical excipients or administration vehicles.

In a particular embodiment, the therapeutic agents may be administered simultaneously, separately, or sequentially in the treatment of a disease, in particular in the treatment of a cancer.

In a particular embodiment, it is provided a fusion molecule as defined in any embodiment of the invention disclosed herein for the treatment of liquid cancer, in particular lleukemia, Lymphoma or Myeloma, or for the treatment of solid cancer, in particular for the treatment of colorectal cancer or adenocarcinoma.

In a particular embodiment, it is provided a fusion molecule as defined in any embodiment of the invention disclosed herein in combination with cyclophosphamide. In a particular embodiment, it is provided a fusion molecule as defined in any embodiment of the invention disclosed herein in combination with cyclophosphamide, for treating cancer, in particular liquid cancer, more particularly lleukemia, Lymphoma or Myeloma, or in particular of solid cancer, more particularly for treating colorectal cancer.

In a particular embodiment of the invention, it is provided a fusion molecule as defined in any embodiment of the invention disclosed herein that inhibits the C-type lectin-like receptor-1 (CLEC-1) signaling pathway, wherein the compound binds to CLEC1, or reduces the expression of functional CLEC1 or is a functional equivalent of CLEC1, in particular an antagonist fusion protein of the binding between CLEC-1 and CLEC-1Lthat binds to CLEC1, more particularly binds to human CLEC1, for use in the treatment of a subject, in particular a human subject, suffering from a cancer with CLEC-1L-positive tumor cells or CLEC-1-positive tumor cells, preferably said fusion protein increases the phagocytosis capability of myeloid cells, in particular dendritic cells and/or macrophages, in particular which increases the phagocytosis of CLEC-1L-positive cells, more particularly of CLEC-1L-positive tumor cells and/or secondary necrotic cells, by dendritic cells and/or macrophages, in particular which increases the phagocytosis of tumor cells and/or secondary necrotic cells by dendritic cells and/or macrophages.

In a particular embodiment of the invention, it is provided a fusion molecule, said fusion molecule enhancing the phagocytosis capability of myeloid cells, in particular dendritic cells and/or macrophages, in particular the phagocytosis of CLEC-1L-positive cells or CLEC-1-positive cell, more particularly of CLEC-1L-positive tumor cells and/or secondary necrotic cells and/or CLEC-1-positive tumor cells, by dendritic cells and/or macrophages, more particularly the phagocytosis of tumor cells and/or secondary necrotic cells by dendritic cells and/or macrophages,

for use in the treatment of a patient suffering from a cancer, or an infectious disease, or sepsis, an autoimmune disease, or an inflammatory disease, including acute or chronic inflammatory diseases, more preferably a cancer, in particular a liquid cancer or a solid cancer, in particular cancer with CLEC-1L-positive tumor cells and/or CLEC-1-positive tumor cells, cancers with glioma cells, breast cancer, hepatocellular carcinoma, lymphoma, more particularly B-cell lymphoma, colon cancer, thyroid cancer, liver cancer, testicular cancer, renal cancer, melanoma, colorectal cancer, adenocarcinoma, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease.

### Method for Treating a Patient

In a further aspect, the invention thus relates to a method for treating a human patient diagnosed with a cancer, or an infectious disease, or sepsis, an autoimmune disease, or an inflammatory disease, including acute or chronic inflammatory diseases, more preferably a cancer, in particular a liquid cancer or a solid cancer, said method comprising administering to the subject a therapeutically effective amount of a fusion molecule according to any embodiment disclosed herein. The fusion molecule may be administered in combination with a conventional treatment, for example with cyclophosphamide.

In a further aspect, the invention thus relates to a method for treating a human patient diagnosed with a cancer, said method comprising:
Assessing if the patient has tumor cells expression CLEC-1Land/or CLEC-1;
When the patient has CLEC-1L-positive tumor cells and/or CLEC-1-positive tumor cells, administer a fusion molecule according to any embodiment disclosed herein.

In a further aspect, the invention thus relates to a method for treating cancer, in a human patient in need thereof, comprising administering to the subject a therapeutically effective amount of a fusion molecule as defined herein. The fusion molecule may be administered in combination with a conventional treatment. As used herein, the term “standard or conventional treatment” refers to any treatment of cancer (drug, radiotherapy, etc) usually administrated to a subject who suffers from cancer. In particular, the fusion molecule of the invention is used in combination with a chemotherapeutic agent, a radiotherapy agent, an immunotherapeutic agent (such as a tumor-targeting monoclonal antibody), a cell therapy agent (such as CAR-T cells), an immunosuppressive agent, a pro-apoptotic agent, an antibiotic, a targeted cancer therapy, and/or a probiotic.

In another aspect, it is provided a fusion molecule according to any embodiment disclosed herein for the treatment of a patient who has CLEC-1-ligand positive tumor cells (i.e; cells expressing a ligand of CLEC-1).

In another aspect, it is provided a fusion molecule according to any embodiment disclosed herein for the treatment of a patient who has tumor cells recognized by CLEC-1, in particular recognized by an anti-CLEC-1 antibody or a Fc-CLEC-1, more particularly a Fc-CLEC-1 fusion molecule according to the invention.

In another aspect, it is provided a fusion molecule according to any embodiment disclosed herein for the treatment of a patient who is or has been treated by a conventional treatment against cancer.

In another aspect, it is provided a fusion molecule according to any embodiment disclosed herein for the treatment of a patient who has cancer and who is treated or has been treated with an agent selected from the group consisting of a chemotherapeutic agent, a targeted cancer therapy, an immunotherapeutic agent or radiotherapy agent.

In another aspect, it is provided a fusion molecule according to any embodiment disclosed herein for the treatment of a patient who has cancer and who is treated or has been treated with an agent selected from the group consisting of a cytotoxic agent, an anti-angiogenic agent, an anti-cancer agent, a cell-cycle/control apoptosis regulating agent, an anti-cancer antibody and a hormonal regulating agent.

In another aspect, it is provided a fusion molecule according to any embodiment disclosed herein for use in the manufacture of a medicament for treating a patient having a cancer, or an infectious disease, or sepsis, an autoimmune disease, or an inflammatory disease, including acute or chronic inflammatory diseases, in particular a liquid cancer, more particularly lleukemia, Lymphoma or Myeloma, or a solid cancer, more particularly colorectal cancer or adenocarcinoma. In another aspect, it is provided a fusion molecule according to any embodiment disclosed herein for use in the manufacture of a medicament for treating cancer, in particular a liquid cancer more particularly lleukemia, Lymphoma or Myeloma, or a solid cancer, more particularly colorectal cancer or adenocarcinoma.

Further aspects and features of the invention will be found in the following examples and in the figures.

The following Figures and Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Legend of the Figures

Some of the figures, to which the present application refers, are in color. The application as filed contains the color print-out of the figures in an archive, which can therefore be accessed by inspection of the file of the application at the patent office.

FIG. 1: Schematic representation of different Fc-CLEC-1 fusion molecules according to the invention. A represents prior Fc-CLEC which comprises 2 identical Fc chains (blue rectangles), and two binding moieties comprising the extracellular domain of CLEC-1 (green spheres). B represents a heterodimeric Fc-CLEC-1 of the invention comprising 2 different Fc chains (blue rectangles with red triangles), and a single binding moiety comprising the extracellular domain of CLEC-1 (green sphere). C represents a heterodimeric Fc-CLEC-1 of the invention comprising 2 different Fc chains (blue rectangles with red triangles), a single binding moiety comprising the extracellular domain of CLEC-1 (green sphere), and another binding moiety corresponding to the extracellular domain of another protein than CLEC-1 (blue sphere). D. represents a heterodimeric Fc-CLEC-1 of the invention comprising 2 different Fc chains (blue rectangles with red triangles), a single binding moiety comprising the extracellular domain of CLEC-1 (green sphere), associated to the C-terminal end of one Fc chain, and an antigen-binding domain associated to the N-terminal domain of a Fc chain (blue and cyan rectangle). E represents a heterodimeric Fc-CLEC-1 of the invention comprising 2 different Fc chains (blue rectangles with red triangles), a single binding moiety comprising the extracellular domain of CLEC-1 (green sphere), another binding moiety corresponding to the extracellular domain of another protein than CLEC-1 (blue sphere), and a single antigen-binding domain associated to a single N-terminal domain of a Fc chain (blue and cyan rectangles). F. represents a heterodimeric Fc-CLEC-1 of the invention comprising 2 different Fc chains (blue rectangles with red triangles), a single binding moiety comprising the extracellular domain of CLEC-1 (green sphere), and two antigen-binding domains, each associated to a single N-terminal domain of a Fc chain (blue and cyan rectangles). G. represents a heterodimeric Fc-CLEC-of the invention comprising 2 different Fc chains (blue rectangles with red triangles), a single binding moiety comprising the extracellular domain of CLEC-1 (green sphere), another binding moiety corresponding to the extracellular domain of another protein than CLEC-1 (blue sphere), each associated to the C-terminal end of a Fc chain, and two antigen-binding domain each associated to a single N-terminal domain of a Fc chain (blue and cyan rectangles). H. represents a heterodimeric Fc-CLEC-of the invention comprising 2 different Fc chains (blue rectangles with red triangles), a single binding moiety comprising the extracellular domain of CLEC-1 (green sphere), another binding moiety corresponding to the extracellular domain of another protein than CLEC-1 (blue sphere), each associated to the C-terminal end of a Fc chain, and two different antigen-binding domain each associated to a single N-terminal domain of a Fc chain (blue, cyan, and red rectangles).

FIG. 2: Anti-tumor effect of Fc CLEC-1 treatment. A. Tumor volume and B. survival of MC38 colon adenocarcinoma engrafted mice following combinatorial treatment of Cyclophosphamide (CPA) and Fc control or Fc CLEC-1 fusion proteins.

FIG. 3: Toxicity scores following intravenous injection of different Fc CLEC-1 formats. A. Toxicity scores were evaluated through time following intravenous injection of either human Fc CLEC-1 (mouse protein), mouse Fc CLEC-1 (human protein), and two different Fc KIH CLEC-1 (Fc-KIH G1 (Hole)-(G4S)3-CLEC1 and Fc-KIH-G1 (knob)-(G4S)3-CLEC1) according to the invention (human protein), according to the table shown in B.

FIG. 4: Analytical SEC profiles (evaluation of monomers, dimers and aggregates forms) of different Fc CLEC-1 or control recombinant proteins (A) and different formulation (B).

FIG. 5: Elisa binding to CLEC-1 ligand (CLEC-1L). Binding of human Fc CLEC-1, Fc-KIH-CLEC1 recombinant proteins and Fc-KIH control to human CLEC-1 ligand

FIG. 6: Pharmacokinetic profiles of Fc CLEC-1 and three Fc-KIH-CLEC1 recombinant proteins according to the invention in mice.

FIG. 7: ELISA binding assay of anti-CLEC-1A antibody with three different Fc KIH CLEC-1 constructs of the invention. Binding of anti-CLEC-1 antibodies to immobilized Fc KIH CLEC-1 and isotype control.

FIG. 8: Inflammatory cytokine secretion. Pro-inflammatory/Pro-tumoral murine IL-6 secretion following Fc CLEC-1 and Fc-KIH-CLEC1 recombinant proteins intravenous injections, as measured by ELISA in mouse serum.

FIG. 9: Example of binding of Fc KIH CLEC-1 construct vs control Fc KIH to tumor cells (here MC38 murine colon adenocarcinoma cells).

FIG. 10: Binding of Fc-CLEC-1 to different tumor cells. Human non-small-cell lung carcinoma (NSCLC) (A549), triple negative breast cancer (SK-BR3), colorectal cancer (CRC) (DLD-1, HT-29), Ovarian (HeLa), hepatocarcinoma (HCC) (HepG2, Huh7), osteosarcoma (U2OS), glioblastoma (U373), T-ALL (HPB-ALL, DND41, Jurkat), lymphoma (T2), B-ALL (Raji, Ramos, RPMI8866), myeloma (U266, RPMI8226), AML (THP-1, U937) were killed by treatment with A. UV-C light (CL-1000 UV Crosslinker, Analytik Jena) (150mJ/cm²), B. X-ray (Faxitron CP 160 (Faxitron X-Ray Corp., Wheeling)) (10 Gy) or chemotherapies C. cisplatin (20 uM Merck), D. staurosporine (1 μM Sigma-Aldrich) and were subsequently incubated in culture conditions for 18 h at 37° C. Then, cells were connected and stained using Fixable Viability Dye (eBioscience) and Fc-CLEC-1 (or Fc-Ctrl) at 100 nM 30 min at 4° C. Percentage of Fc-CLEC1 positive cells was determined after reading on a cytometer.

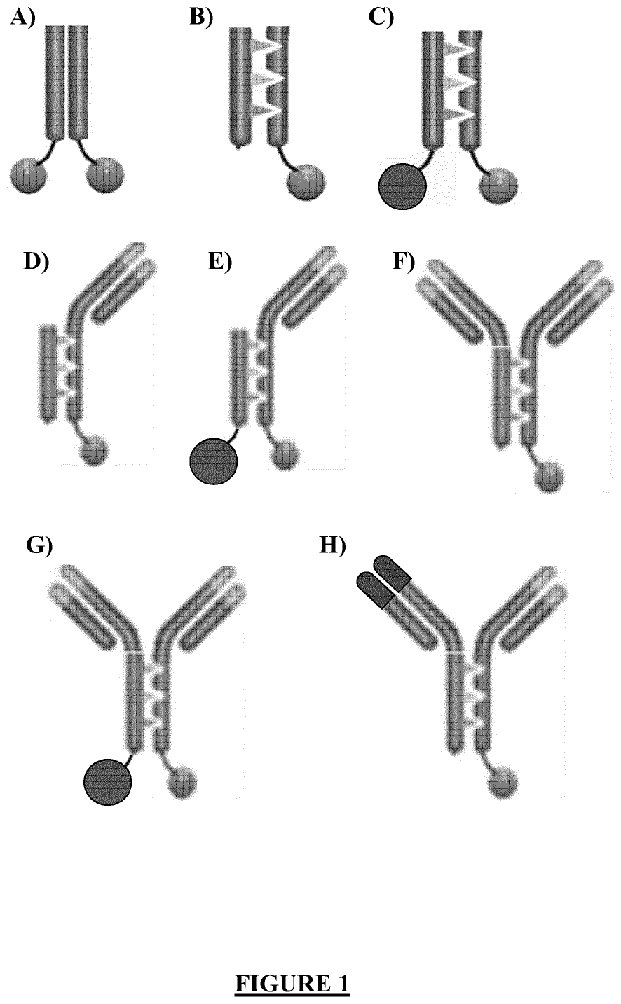

### Material and Methods

### Tumor Model and Treatment:

MC38 colon adenocarcinoma cells (ATCC, Manassas, Virginia, USA), were cultured according to the manufacturer instructions in 1640 RPMI medium (Life Technologies), supplemented with (10% endotoxin-free fetal calf serum (Thermo Fisher), 2 mM L-glutamine (Sigma-Aldrich), 100U/ml Penicillicine; 100 μg/ml Streptamycine (Life Technologies, Carlsbad, California, USA) at 37° C. and 5% CO2. 1 million cells/mouse were sc. Injected in the left flank of C57/B16 mice (Janvier labs, Le Genest-Saint-Isle, France). Mice were injected with Cyclophosphamide (CPA) (150 mg/kg, Sigma-Adrich, St. Louis, Missouri, USA) when tumor diameter reached 65 mm3 (at 7-12d) and co-injected with mouse Fc control or Fc CLEC-1 fusion proteins (5 mg/kg, twice a week for 3 weeks). Tumor growth was measured blind with caliper and expressed as the area based on two perpendicular diameters ((length×width)1.5)×π/6. Mice were euthanized when tumor size reached the permitted size.

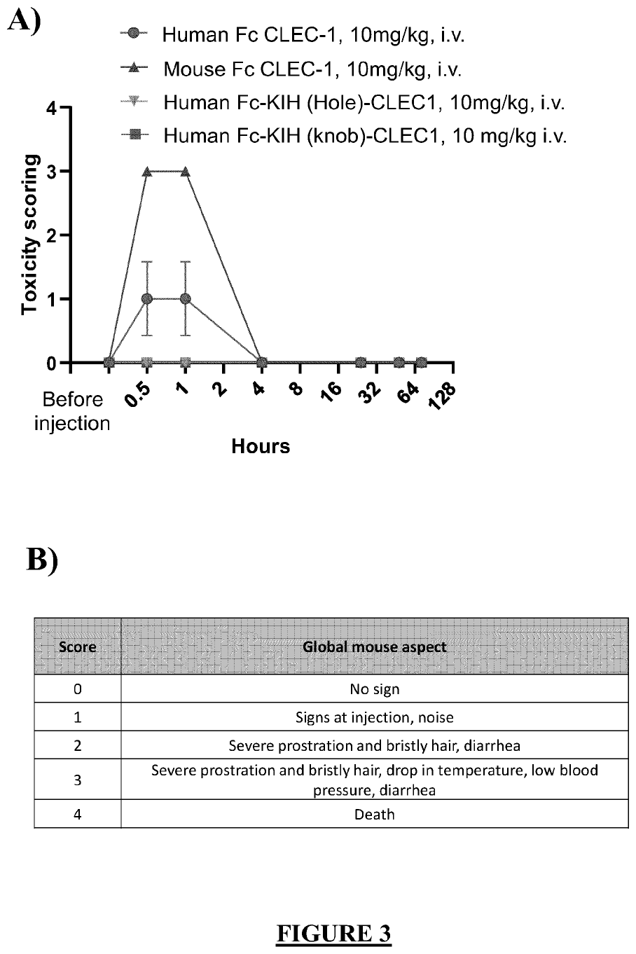

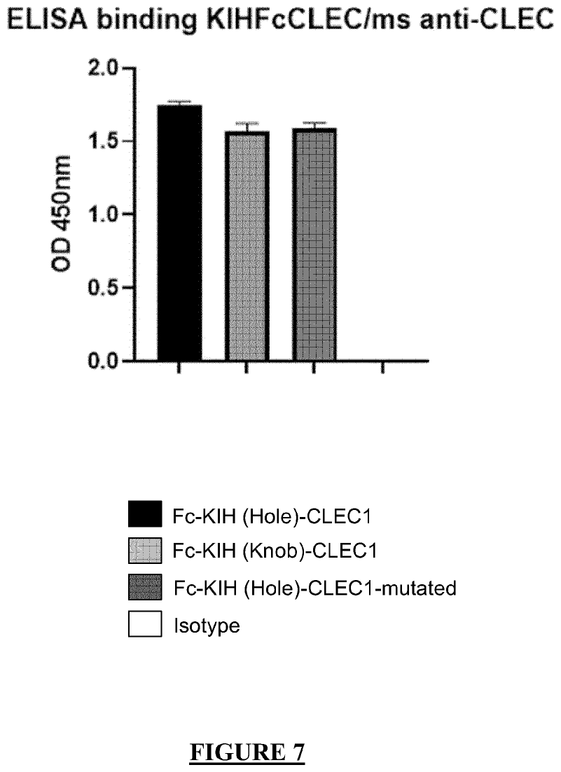

Pharmacokinetic and in vivo toxicity profiling of Fc CLEC-1 recombinant proteins: Fc CLEC-1 recombinant proteins were injected intravenously in the caudal vein at 10 mg/kg in naïve Balb-c mice. Blood was collected following injection at different time points as listed in FIG. 7. A close monitoring of signs of toxicity was performed through time, based on the scoring methodology detailed in FIG. 3B. Pharmacokinetic profiling was performed using a sandwich ELISA assay. For ELISA assay, goat anti-human Fc (Jackson immunoresearch reference 109-005-098) was immobilized on plastic at 1 μg/ml and serum or standard recombinant protein were added to quantify concentration in serum. After incubation and washing, peroxidase-labeled donkey anti-human IgG (Jackson immunoresearch reference 709-035-149) was added and revealed by conventional methods.

### Analytical SEC Profiling of Fc CLEC-1 Recombinant Proteins:

SEC profile was performed by gel filtration chromatography (GeHealthcare Superdex200 10/300GL column) using AktaPüre system. Antibody was injected in loop (100 μl) with PBS buffer and analysed by AktaPüre software.

### ELISA IL6 Assays:

Murine IL-6 detection in mouse serum was performed using Mouse IL-6 ELISA Set (BD Biosciences, Franklin Lakes, New Jersey, USA, OptEIA-555240), following the manufacturer's instructions.

### ELISA Binding CLEC-1L-CLEC-1 Assays:

Binding of different Fc CLEC-1 recombinant proteins or Fc controls to human CLEC-1L recombinant protein was performed using CLEC-1L binding ELISA. For activity ELISA assay, recombinant hCLEC-1L-His (Biotechne; reference 18010-H07B) was immobilized on plastic at 2 μg/ml in P96 polysorp plate and purified recombinant proteins were added to measure binding. After incubation and washing, peroxidase-labeled donkey anti-human IgG (Jackson immunoresearch reference 709-035-149) was added and revealed by conventional methods.

ELISA Binding Assay Anti-CLEC-1A with Coated Fc KIH CLEC-1A

For binding ELISA assay, human Fc KIH CLEC-1A molecules were immobilized on plastic at 1 μg/ml and mouse anti-CLEC-1A monoclonal antibody (OSE Immunotherapeutics) was added to measure binding at 5 μg/ml. After incubation and washing, peroxidase-labeled donkey anti-mouse IgG (Jackson immunoresearch reference 715-036-151) was added and revealed by conventional methods.

### Cellular Binding Assays:

Fc KIH or Fc KIH CLEC-1 constructs were coupled with AF647 using A30009 Alexa Fluor™ 647 Microscale Protein Labeling Kit (FISHER, Hampton, New Hampshire, USA). Tumor cells were stained using a PBS BSA 1% Azide 0.02% solution at pH6, with fusion proteins at 200 nM each for 45 min on ice.

### Example 1: Anti-Tumor Effect of a Fc-CLEC-1 Fusion Molecule

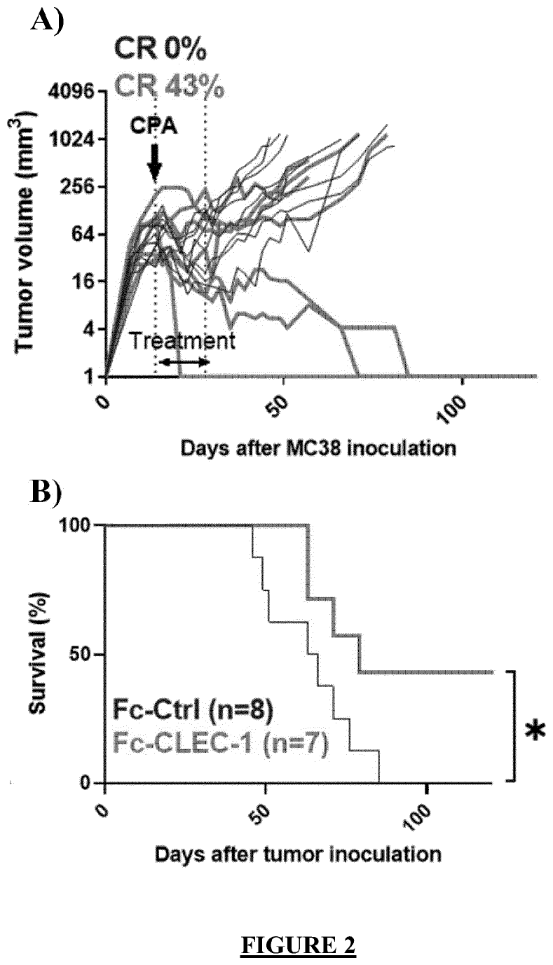

FIG. 2 illustrates results obtained in a treatment of a cancer model highly relevant for oncology. As illustrated, a Fc-CLEC-1 fusion molecule potentiates anti-tumor effects of chemotherapy. For instance, in the MC38 colon adenocarcinoma syngeneic mouse model, Fc CLEC-1 treatment but not control Fc recombinant treatment improves the positive effect of cyclophosphamide (CPA) on tumor shrinkage (FIG. 2A) and mouse survival (FIG. 2B).

### Example 2: In Vivo Toxicity Though Adverse Effects Analysis

To choose the most potent or safest Fc CLEC-1 fusion protein format for oncology applications, prior art and Fc KIH CLEC-1 fusion proteins were evaluated in vivo. Prior art Fc CLEC-1 molecules induced transient signs of toxicity (adverse effects) in animals treated with these molecules by intravenous administration (using either mouse or human CLEC-1 proteins), whereas the Fc KIH CLEC-1 formats did not induce any sign of toxicity in vivo (FIG. 3A), as monitored by careful scoring of global mouse aspect (FIG. 3B). Fc-KIH G1 (Hole)-(G4S)3-CLEC1 and Fc-KIH-G1 (knob)-(G4S)3-CLEC1 which represent two embodiments of the invention wherein a binding moiety is fused to the C-terminal end of the hole or knob polypeptide. Thus, while the administration of prior art Fc-CLEC cannot be considered to be toxic when reasonable doses are administered (adverse effects may occur when a prescribed drug is administered to a patient in need thereof), the heterodimeric Fc-CLEC-1 fusion molecule of the present invention initiates almost no adverse effects, which is a clear advantage over the prior art compounds that cause adverse effects as illustrated with the Fc-CLEC-1 compounds on FIG. 3A.

### Example 3: Aggregation of Fc-CLEC Fusion Molecules

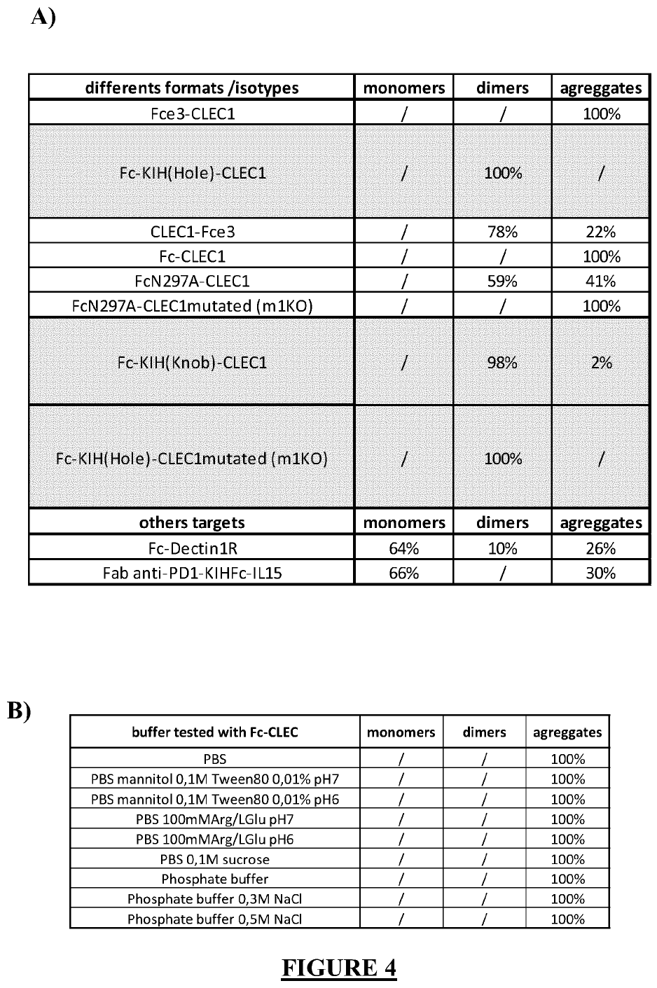

Analytical SEC profiling of prior art Fc CLEC-1 fusion protein reveals the presence of large aggregates whereas the analysis of the Fc KIH CLEC-1 fusion proteins of the invention reveals that, surprisingly, this molecule forms dimers without any aggregates A summary table of the different types of isotype fused to proteins evaluated by analytical SEC is shown in FIG. 4, highlighting the fact that the Fc KIH CLEC-1 format (in grey) of the invention is the only Fc fusion format enabling absence of aggregates. This aggregability of Fc CLEC-1 was specific to recombinant CLEC-1 protein, because another target, like Fc-Dectin-1R for example, did not induce as much dimers/aggregates. Besides, and as illustrated, several, different Fc KIH CLEC-1 according to the invention (Fc-KIH-G1 (hole)-(G4S)3-CLEC-1, comprising or consisting of a chain comprising or consisting of SEQ ID No. 5 and a chain comprising or consisting of SEQ ID No. 3; Fc-KIH-G1 (knob)-(G4S)3-CLEC-1 comprising or consisting of a chain comprising or consisting of SEQ ID No. 23 (or SEQ ID No. 3+SEQ ID No. 13+SEQ ID No. 2) and a chain comprising or consisting of SEQ ID No. 4, and Fc-KIH-G1 (hole)-(G4S)3-mutatedCLEC-1, comprising a chain comprising or consisting of SEQ ID No. 4+SEQ ID No. 13+SEQ ID No. 19 and a chain comprising or consisting of SEQ ID No. 3)—are the only one that do not form aggregate. In fact, the heterodimeric Fc-IL-15 fusion molecule (i.e, wherein the binding moiety issued from CLEC-1 is substituted for IL-15 in the fusion molecule) form aggregates (see the lower lines of the table on FIG. 4). Thus, the inability to form aggregate is only achieved with heterodimeric Fc-CLEC-1 fusion molecules of the invention. Different classical formulations were tested to prevent aggregation, as mentioned in FIG. 4, but any buffer might be efficient to reduce aggregation in Fc CLEC-1. Absence of aggregates of the heterodimeric Fc-CLEC-1 fusion molecules of the present invention might underlie the lack of in vivo toxicity specifically observed following treatment with said molecules. As illustrated in FIG. 4, dimers of heterodimeric Fc-KIH-CLEC fusion proteins were obtained. The authors expected to obtain monomers of heterodimeric Fc-based monomeric proteins, as thought by the prior art (see for example Ha J-H, 2016, Front. Immunol 7:394); indeed, natural homodimeric proteins, like Dectin-1 or IL-15, when only one monomer is associated to heterodimeric immunoglobulin led to monomers of heterodimeric Fc-monomeric proteins, as illustrated on FIG. 4A: Fc-dectin1R and Fab anti-PD1-KIH-Fc-IL15 fusion proteins are almost only or only monomers. In the constructions of the invention illustrated in this example, the authors associated a single CLEC-1 domain to the heterodimeric immunoglobulin. Thus, while the CLEC-1 domain is only present on one of the Fc polypeptide chains of the constructs of the invention, dimers of the fusion proteins of the invention are dimers and not monomers are obtained. These dimers did not form large aggregates, on the contrary to other formats of Fc CLEC-1 or other Fc proteins: Fc CLEC-1, Fc-dectin1R and Fab anti-PD1-KIH-Fc-IL15 fusion proteins aggregate. The fusion proteins of the invention form dimers, which was unexpected, and do not form large aggregate.

### Example 4. Binding of Fc-CLEC-1 Fusion Molecule According to the Invention to a Known Ligand of CLEC-1, CLEC-1L

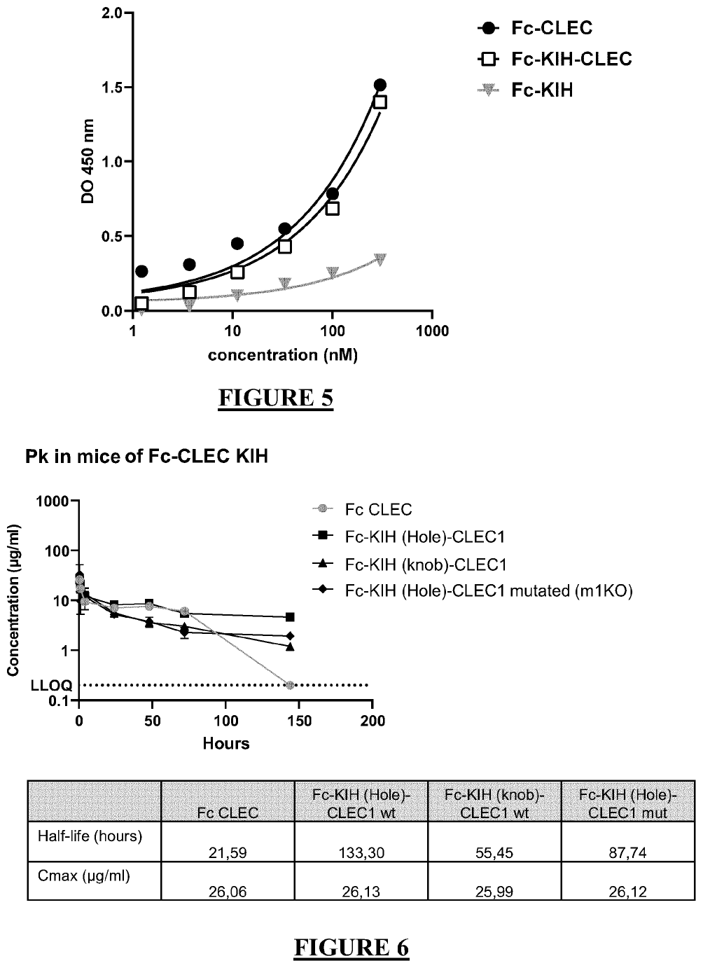

Human CLEC-1L is a ligand of human CLEC-1 (unpublished results). To validate the functionality of several, different Fc KIH CLEC-1 fusion proteins according to the invention, we tested their binding capacity to CLEC-1L by ELISA assay (FIG. 5) and found that the binding of the different Fc KIH CLEC-1 to the natural ligand of CLEC-1 (CLEC-1L) is conserved. It is of importance to highlight that this capability is observed irrespectively of the binding of the CLEC-binding moiety to the hole or knob chain, and irrespectively of the presence of the full extracellular domain of CLEC-1 or a mutated version thereof). Next, the pharmacokinetics profile of prior art Fc CLEC-1 versus Fc KIH CLEC-1 formats have been evaluated. The Fc KIH CLEC-1 formats are cleared less rapidly that Fc CLEC-1 in mouse serum following an intravenous injection at 10 mg/kg (FIG. 6). The half-life of the Fc KIH CLEC-1 formats of the invention is at least twice the length of the half-life of Fc-CLEC-1. Further, while the in vivo concentration of Fc-CLEC-1 falls after 72 hours, the Fc KIH CLEC-1 constructs of the invention remain at high concentration in the serum of treated mice longer, and even 6 days after injection, whereas it is entirely cleared in its aggregated Fc CLEC-1 format at this same time point.

### Example 5. Binding of Anti-CLEC-1 Antibody to Fc KIH CLEC-1 Constructs

To evaluate the conservation of the structure of CLEC-1 when fused to the Fc KIH domain, anti-CLEC-1 mouse antibody has been used to target immobilized several, different Fc KIH CLEC-1 proteins according to the invention. As illustrated on FIG. 7, anti-CLEC-1 mouse antibody recognizes CLEC-1 irrespectively of its binding to either the hole chain or knob chain. Further, the anti-CLEC-1 mouse antibody also recognizes mutated version of CLEC-1, in particular truncated version of CLEC-1 wherein the N-terminal end of the extracellular domain of CLEC-1 has been deleted. These results illustrate that CLEC-1 when fused to the Fc KIH keeps its structural conformation, allowing in vivo or in vitro interaction between the Fc KIH CLEC-1 of the invention with the ligands of CLEC-1.

### Example 6. Absence of Inflammatory Effect Initiated by the Heterodimeric Fc-CLEC-1 Fusion Protein of the Invention

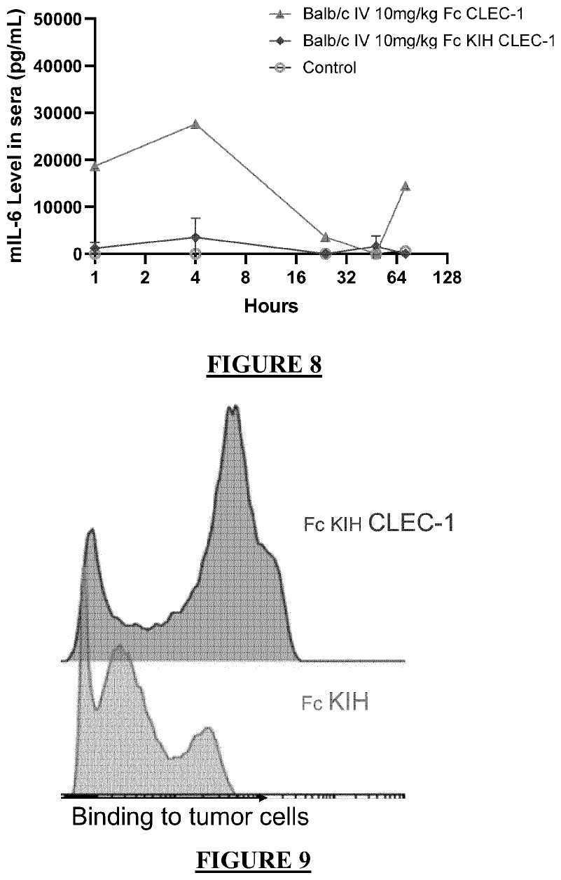

To evaluate the undesirable pro-tumoral/pro-inflammatory effects of Fc CLEC-1 molecules in vivo, we investigated the impact of Fc CLEC-1 injection on IL-6 secretion in mouse serum. We found that while prior art aggregated Fc CLEC-1 fusion proteins induced IL-6 secretion in serum, the heterodimeric Fc KIH CLEC-1 proteins of the invention did not induce IL-6 secretion (FIG. 8), thereby validating this format as being safer for oncology applications.

### Example 7. Capability for the Heterodimeric Fc-CLEC-1 Fusion Protein of the Invention to Bind to Tumor Cells Expressing a Ligand of CLEC-1

To evaluate the capacity of the heterodimeric Fc KIH CLEC-1 construct of the invention to target tumor cells, we carried out a cellular binding assay of Fc KIH (control) vs heterodimeric Fc KIH CLEC-1 on MC38 murine colon adenocarcinoma cells (FIG. 9), thereby validating that this format binds CLEC-1 ligand positive tumor cells.

### Example 8. Binding of Fc-CLEC-1 Fusion Molecule

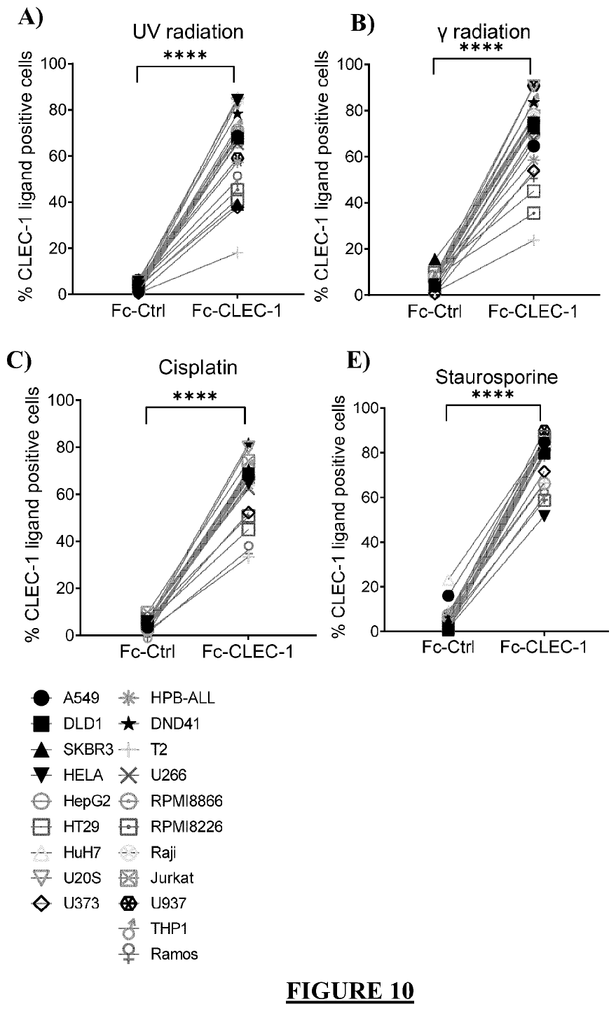

As illustrated on FIG. 10, Fc-CLEC-1 is able to bind to several, different, tumor cells treated by conventional methods against cancer (more than 21 tumor cell lines are tested). These results illustrate the capability of Fc-CLEC-1 fusion molecules to bind to different tumor cells, thereby sustaining a broad use of the fusion molecules of the invention in the treatment of different types of cancer.

1. A fusion molecule comprising:
(i) At least one binding moiety comprising or consisting of at least a portion of the extracellular domain of human C-type lectin domain family 1 member A (CLEC-1), wherein the at least portion of the extracellular domain of human CLEC-1 is a functional equivalent of the extracellular domain of wild type human CLEC-1, and
(ii) a heterodimeric immunoglobulin or fragment thereof comprising:
a. a first Fc polypeptide chain comprising a first CH3 domain (CH3B chain or hole chain), and
b. a second Fc polypeptide chain comprising a second CH3 domain (CH3A chain or knob chain),
wherein the first and second CH3 domains are different,
one of the at least one binding moiety being fused to the C-terminal end or the N-terminal end of the first Fc polypeptide, and/or one of the at least one binding moiety being fused to the C-terminal end or N-terminal end of the second Fc polypeptide chain.

2. The fusion molecule according to claim 1, wherein the CH3 domains of the first Fc polypeptide chain and of the second Fc polypeptide are selected from the group of the CH3 domains referenced KiH, KiH_{S-S}, HA-TF, ZW1, 7.8.90, DD-KK, EW-RVT, SEED and A107, more particularly wherein the first Fc polypeptide has the amino acid residues set forth in SEQ ID No. 3 and the second Fc polypeptide chain has the amino acid residues set forth in SEQ ID No. 5, or wherein the first Fc polypeptide has the amino acid residues set forth in SEQ ID No. 10 and the second Fc polypeptide chain has the amino acid residues set forth in SEQ ID No. 12.

3. The fusion molecule according to claim 1 or 2, wherein a single binding moiety is present, the binding moiety being fused to either the C-terminal end of the first Fc polypeptide chain or the C-terminal end of the second Fc polypeptide chain.

4. The fusion molecule according to any one of claims 1 to 3, wherein a binding moiety is fused to the C-terminal end or the N-terminal end of the first Fc polypeptide chain, in particular the C-terminal end of the first Fc polypeptide chain, and wherein another binding molecule, different from the binding moiety associated the first Fc polypeptide chain, is fused to the C-terminal end or the N-terminal end of the second Fc polypeptide chain, in particular when the at least one binding moiety is present at one end of the same Fc polypeptide chain, the binding molecule is at the opposite end of the Fc polypeptide chain.

5. The fusion molecule according to any one of claims 1 to 4, wherein the at least one binding moiety is fused to the Fc polypeptide chain through a linker peptide, more particularly through a linker peptide of SEQ ID No. 13.

6. The fusion molecule according to any one of claims 1 to 5, wherein the first Fc polypeptide chain, or the second Fc polypeptide chain, or both the first and the second Fc polypeptide chains comprise(s) a CH2 domain, in particular at the N-terminal end of the CH3 domain, in particular wherein the CH2 domains are different.

7. The fusion molecule according to any one of claims 1 to 6, wherein the first Fc polypeptide chain, or the second Fc polypeptide chain, or both the first and the second Fc polypeptide chains comprise(s) a hinge region, in particular at the N-terminal end of the Fc polypeptide chain, more particularly at the N-terminal end of the CH2 domain when present.

8. The fusion molecule according to any one of claims 1 to 7, wherein:
the first Fc polypeptide chain comprises from its N-terminal end to its C-terminal end: a hinge region, a CH2 domain and a first CH3 domain (CH3B chain or hole chain), respectively, and
the second Fc polypeptide chain comprises from its N-terminal end to its C-terminal end: a hinge region, a CH2 domain, a second CH3 domain (CH3B chain or hole chain), respectively,
the at least one binding moiety being fused through a linker to the C-terminal end of the first CH3 domain or the second CH3 domain.

9. The fusion molecule according to any one of claims 1 to 8, wherein the at least one binding moiety is fused to the C-terminal end of the first Fc polypeptide chain, in particular through a linker peptide of SEQ ID No. 13.

10. The fusion molecule according to any one of claims 1 to 8, wherein the at least one binding moiety is fused to the C-terminal end of the second Fc polypeptide chain, in particular through a linker peptide of SEQ ID No. 13.

11. The fusion molecule according to any one of claims 1 to 10, wherein the portion of the extracellular domain of human CLEC-1 corresponds to a mutated extracellular domain of CLEC-1, in particular of SEQ ID No. 2.

12. The fusion molecule according to claim 11, wherein the mutated extracellular domain of CLEC-1 has the sequence of amino acid residues set forth in SEQ ID No. 19.

13. The fusion molecule according to any one of claims 1 to 12, which comprises a first Fc polypeptide chain comprising of consisting of the amino acid sequence set forth in SEQ ID No. 4 and a second Fc polypeptide chain comprising of the amino acid sequence set forth in SEQ ID No. 3.

14. The fusion molecule according to any one of claims 1 to 13, which comprises a first Fc polypeptide chain comprising of consisting of the amino acid sequence set forth in SEQ ID No. 5 and a second Fc polypeptide chain comprising of the amino acid sequence set forth in SEQ ID No. 3.

15. The fusion molecule according to any one of claims 1 to 13, which comprises a first Fc polypeptide chain comprising of consisting of the amino acid sequence set forth in SEQ ID No. 4 and a second Fc polypeptide chain comprising of the amino acid sequence set forth in SEQ ID No. 23.

16. The fusion molecule according to any one of claims 1 to 15, further comprising:
(iii) At least one antigen-binding domain comprising or consisting of a variable domain of an antibody or antigen-binding fragment thereof, said antigen-binding domain being fused to either the first Fc polypeptide chain or the second Fc polypeptide chain, or a first antigen-binding domain being fused to the first Fc polypeptide chain and a second antigen-binding domain being fused to the second Fc polypeptide chain, in particular when the at least one binding moiety is present at one end of the same Fc polypeptide chain, the antigen-binding domain is at the opposite end of the Fc polypeptide chain.

17. The fusion molecule according to claim 16, wherein the antigen-binding domain specifically binds to an antigen expressed by macrophages, and/or lymphocytes and/or tumor cells, in particular selected from the group consisting of SIRPalpha, SIRPbeta, SIRPgamma, CD47, CTLA-4, CD86 (B7.2), CD28, CD40, CD40L, ICOS, ICOS-L, OX40L, GITR, HVEM, BTLA, CD160, LIGHT, TNFRSF25, 2B4, CD48, Tim1, Tim3, Tim4, Gal9, LAG-3, CD40, CD40L, CD70, CD27, VISTA, B7H3, B7H4 (B7x), TIGIT, CD112, HHLA2 (B7-H7), TMIGD2 (CD28H), Butyrophilin-like2 (BTNL2), SIGLEC, AXL, B7.1, B7-DC, B7-H1, B7-H2, B7-H3, B7-H4, CD19, CD20, CD22, CD24, CD137 (4-1BB), CD137L (4-1BBL), CEA, CXCR3, CXCR4, EGFR, EGFRvIII, ELTD1, EMR1, EMR2, EMR3, EMR4P, ENG, EPCAM, EPHR, PD-L1, TLR1, TLR10, TLR2, TLR3, TLR4, VEGFR, VEGFR2, VIPR1, VIPR2, CD101, CD3, CD30, CD38, CD39, CD44, DR3, LFA-1, NKG2D, PD-1, and PDL2.

18. The fusion molecule according to claim 16 or 17, wherein the antigen-binding domain is fused to the N-terminal end of either the first Fc polypeptide chain or the second Fc polypeptide chain, or wherein the first antigen-binding domain is fused to the N-terminal end of the first Fc polypeptide chain and the second antigen-binding domain is fused to the second Fc polypeptide chain, in particular with the N-terminal end of a CH2 domain or, when present, a hinge region.

19. The fusion molecule according to claim 16 or 17, wherein two antigen-binding domains are present, each being associated with a single Fc polypeptide chain, the first antigen-binding domain and the second antigen-binding domain binding to two different epitopes, in particular to two different antigens.

20. The fusion molecule according to any one of claims 1 to 19, for use in the treatment of a patient having a disease selected from a cancer, in particular solid cancer or liquid cancer, or an infectious disease, or sepsis, an autoimmune disease, or an inflammatory disease, including acute or chronic inflammatory diseases.

21. The fusion molecule according to any one of claims 1 to 20 for use in the treatment of a patient who has cancer and who is treated or has been treated with a conventional treatment against cancer, in particular with an agent selected from the group consisting of a chemotherapeutic agent, a targeted cancer therapy, an immunotherapeutic agent or radiotherapy agent, more particularly an agent selected from the group consisting of a cytotoxic agent, an anti-angiogenic agent, an anti-cancer agent, a cell-cycle/control apoptosis regulating agent, an anti-cancer antibody and a hormonal regulating agent, the fusion molecule and the agent being administered either simultaneously, separately or sequentially.

22. A nucleic acid molecule or a combination of nucleic acid molecules encoding a fusion protein according to any one of claims 1 to 21.
